SEQUENCE LISTING (17 sequences)

SEQ ID NO: 1 - RNA, 45 nt, Rat
  misc_feature (32)...(32): Deletion of this residue results in a ribozyme with only antisense activity
gaaggccggguacugaugaguccgugaggacgaaacgucagccau

SEQ ID NO: 2 - RNA, 45 nt, Mouse
gaagaggcugacugaugaguccgugaggacgaaacauaggcaccg

SEQ ID NO: 3 - RNA, 40 nt, Human
gggggaacugaugaguccgugaggacgaaacgucagccau

SEQ ID NO: 4 - RNA, 45 nt, Rat
ggaaagacugaugaguccgugaggacgaaagcagaaagugcaugg

SEQ ID NO: 5 - RNA, 38 nt, Human
gagcagacugaugaguccgugaggacgaaaguucaugg

SEQ ID NO: 6 - RNA, 23 nt, Human and rat
  misc_feature (11)...(12): Ribozyme target site
gggggggaccauggcugacguuu

SEQ ID NO: 7 - DNA, 30 bp, Artificial Sequence
  Synthetic oligonucleotide with catalytic activity as described in the Specification
gtcagccaggctagctacaacgaggtcccc

SEQ ID NO: 8 - DNA, 32 bp, Artificial Sequence
  Synthetic oligonucleotide with catalytic activity as described in the Specification
gtcagccaggctagctacaacgaggtcccccc

SEQ ID NO: 9 - DNA, 36 bp, Artificial Sequence
  Synthetic oligonucleotide with catalytic activity as described in the Specification
aaacgtcagccaggctagctacaacgaggtcccccc

SEQ ID NO: 10 - RNA, 47 nt, Human
gggaaggccgggaacugaugaguccgugaggacgaaacgucagccau

SEQ ID NO: 11 - RNA, 23 nt, Artificial Sequence
  Example of a loop 2 region of hammerhead ribozyme
cugaugaguccgugaggacgaaa

SEQ ID NO: 12 - RNA, 22 nt, Mouse
gauggcugacguuuacccggcc

SEQ ID NO: 13 - DNA, 39 bp, Artificial Sequence
  Synthetic oligonucleotide complementary to SEQ ID NO. 14 and based on mouse TNFa ribozyme target RNA
taatacgactcactatagatggctgacgtttacccggcc

SEQ ID NO: 14 - DNA, 39 bp, Artificial Sequence
  Synthetic oligonucleotide complementary to SEQ ID NO. 13 and based on mouse TNFa ribozyme target RNA
attatgctgagtgatatctaccgtctgcaaatgggccgg

SEQ ID NO: 15 - RNA, 24 nt, Artificial Sequence
  Synthetic oligonucleotide complementary to SEQ ID NO. 14 and based on mouse TNFa ribozyme mouse TNFa short RNA substrate
cggugccuaugucucagccucuuc

SEQ ID NO: 16 - DNA, 30 bp, Rat
ggagtcgttggccgggtaaacgtcagccat

SEQ ID NO: 17 - RNA, 40 nt, Artificial Sequence
  A ribozyme having a GUU target site corresponding to codon 4 of human PKCa mRNA
gggaacugaugaguccgugaggacgaaacgucagccaugg

(19) United States
(12) Patent Application Publication
(10) Pub. No.: US 2003/0045486 A1
(43) Pub. Date: Mar. 6, 2003

(54) Amino-modified ribozymes

(75) Inventor: Mouldy Sioud, Oslo (NO)

Correspondence Address: BAKER & BOTTS, 30 ROCKEFELLER PLAZA, NEW YORK, NY, 10112

(73) Assignee: The Norwegian Radium Hospital Research Foundation

(21) Appl. No.: 09/725,926

(22) Filed: Nov. 30, 2000

Related U.S. Application Data
(63) Continuation of application No. PCT/GB99/01706, filed on May 28, 1999.

(30) Foreign Application Priority Data
Jun. 1, 1998 (GB) GB9811750.0

(51) Int. Cl. A61K 48/00; C07H 21/02

(52) U.S. Cl. 514/44; 536/23.1

(57) ABSTRACT

The invention relates to ribozymes modified to improve their stability, methods for producing such enzymes and the use of the modified enzymes in methods such as therapeutic treatment. In particular, a typical ribozyme according to the invention includes a modified ribozyme, wherein three or more pyrimidine nucleotides in said ribozyme are modified at the 2′-position, wherein said pyrimidine nucleotides are modified to 2′-amino pyrimidine nucleotides and said ribozyme exhibits improved stability to RNAse degradation and exhibits 85% or more catalytic activity of the unmodified ribozyme.

The present invention relates to ribozymes modified to improve their stability, methods for producing such enzymes and the use of the modified enzymes in methods such as therapeutic treatment.

Some RNA is known to have associated catalytic activity. Catalytic RNAs include Group I and Group II introns and ribozymes of the hammerhead, hairpin, and hepatitis delta virus type and the subunit of RNase P. The presence of divalent metal ions, e.g. Mg²⁺ or Mn²⁺ is essential for their activity.

Ribozymes are catalytic oligonucleotide RNAs capable of cleaving other RNA, in particular mRNA or pre-mRNA and thereby disrupting the expression of proteins by cleaving their corresponding transcripts. Such oligonucleotide RNAs are characterized by a conserved portion flanked by short binding site recognition sequences. Since they are specific to particular mRNAs, the expression of particular genes can be blocked without affecting normal functions of other genes. Thus ribozymes are ideal agents for therapeutic interventions against malfunctioning or foreign gene products or for elucidating the functional roles of gene products.

In general, the most difficult step in achieving in vitro or in vivo applications of ribozymes is the delivery of the ribozymes into the cells. There are at present two basic strategies for the delivery of ribozymes into cells and animal, namely endogenous delivery in which a gene encoding the ribozyme is delivered into cells, for example in the form of a DNA vector (Sioud & Drlica, 1991, PNAS, 88, p7307-7307) and exogenous delivery, wherein a presynthesized ribozyme is applied to cells, e.g. via microinjection or transfection, with or without a carrier such as liposomes or CaCl₂ (Sioud et al., 1992, J. Mol. Biol., 223, p831-835). Alternative exogenous delivery methods have relied on the conjugation of the oligonucleotides to polylysine compounds (Leoneti et al., 1990, Bioconjugate Chem., 1, p149-153) or to lipophilic groups, such as cholesterol (MacKellar et al., 1992, Nucl. Acids Res., 20, p3411-3417).

Like all RNA, catalytic RNA is highly susceptible to degradation by RNAses. Thus unmodified ribozymes tend to have low stability in cell culture supernatants containing fetal calf serum. To overcome this problem modified ribozymes have been made in which the 2′-hydroxyl position has been protected by the use of modified ribonucleotides. Almost invariably this has resulted in a loss of catalytic activity.

For example, Pieken et al. (1991, Science, 253, p314-317) prepared various modified hammerhead ribozymes in which all uridine bases were replaced with 2′-fluorouridines or 2′-aminouridines or all cytidines were replaced with 2-fluorocytidine or 2′-aminocytidine, or all pyrimidine bases were replace with corresponding 2′-fluoro or 2′-amino derivatives. Although these modifications conferred stability on the ribozymes to differing extents, only the 2′-fluorocytidine substituted ribozyme retained more than 50% of the catalytic activity of the unmodified ribozyme (wherein catalytic activity=k_{cat}/Kₘ [μM⁻¹min⁻¹]). In particular, where all pyrimidines had been replaced, only 1.9 or 3.8% activity was retained in the 2′-amino or 2′-fluoro derivatized ribozymes, respectively.

Scherr et al. (1997, J. Biol. Chem., 272(22), p14304-4313) performed similar modifications but again was only able to achieve at best 42% of catalytic activity by modifying all pyrimidines to their 2′-fluoro derivatives. This was improved to 65% by replacing 2 specific uridine bases in the conserved region with 2′-aminouridines. Similarly, Heidenreich et al. (1994, J. Biol. Chem, 269(3), p2131-2138) observed that catalytic activity could be improved in ribozymes in which all pyrimidines had been modified to their 2′-fluoro derivatives by the replacement of the 2 specific bases with 2′-aminouridine, with only a small loss of stability. On the basis of the above results, the view in the art, as expressed by Beigelman et al. (1995, J. Biol. Chem., 270(43), p25702-25708) was that a strategy of uniform modification could not be directly applied to ribozymes.

It has however surprisingly been found that the use of 2′-amino modified pyrimidines can provide ribozymes of improved stability which retain substantially the activity of the unmodified ribozyme.

Thus viewed from one aspect the present invention provides a modified ribozyme, wherein three or more pyrimidine nucleotides in said ribozyme are modified at the 2′-position, wherein said pyrimidine nucleotides are modified to 2′-amino pyrimidine nucleotides and said ribozyme exhibits improved stability to RNAse degradation and exhibits 85% or more catalytic activity of the unmodified ribozyme.

Modified ribozymes of the invention may be further derivatized as mentioned below, but are only amino derivatized at the 2′ position of the pyrimidine bases.

Preferably, for example, 5 or more bases are modified. Alternatively viewed, all or substantially all (for example at least 80%, 85%, 90% or 95%, e.g. >90%) of the pyrimidine bases of the ribozymes are 2′-amino modified and more than 20%, e.g greater than 50%, catalytic activity is retained. In other words the ribozyme exhibits 20% or more of the catalytic activity of the corresponding unmodified (i.e. underivatised) ribozyme.

As described herein, catalytic activity may be measured as k_{cat}/Kₘ [μM⁻¹min⁻¹] or k_{cat} (Trevor Palmer in “Understanding enzymes”, 3rd Edition, 1991, p1-399, Ellis Horwood press) or any other appropriate assessment which provides a measurement of the cleavage activity of the modified or unmodified ribozyme. Improved stability as referred to herein refers to an improved half life in one or more aqueous solutions. Conveniently, half life may be measured in serum such as fetal calf serum. Preferably, a greater than 100-fold improvement in half life, especially preferably a greater than 10000-fold improvement, is achieved.

Preferably, all (i.e. global, uniform derivatization) or substantially all (for example at least 80%, 85%, 90% or 95%) of the pyrimidine nucleotides which are present are 2′-amino modified. Furthermore, it is preferable that less than 50% of the bases present in the ribozyme to be modified are pyrimidines. Advantageously, less than 40%, 30% or 20% of the bases in the ribozyme are pyrimidines.

In general, reference to the ribozymes of the invention as used herein may include both DNA and RNA ribozymes and also ribozymes which contain both deoxyribonucleotides and ribonucleotides. Furthermore, any such ribozymes may be capable of cleaving RNA target sequences or composites thereof. For exogenenous delivery of nuclease-resistant ribozymes, small DNA ribozymes may represent an advantageous choice e.g. for gene therapy.

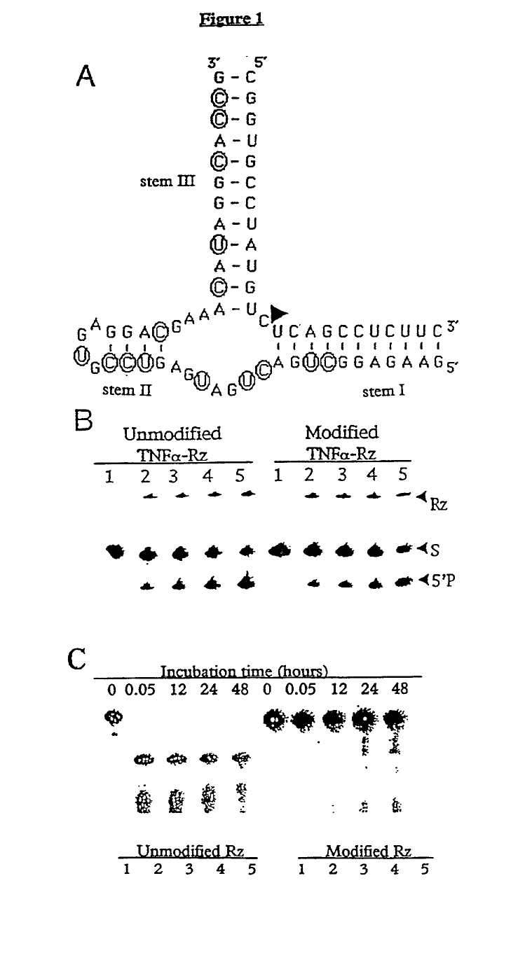

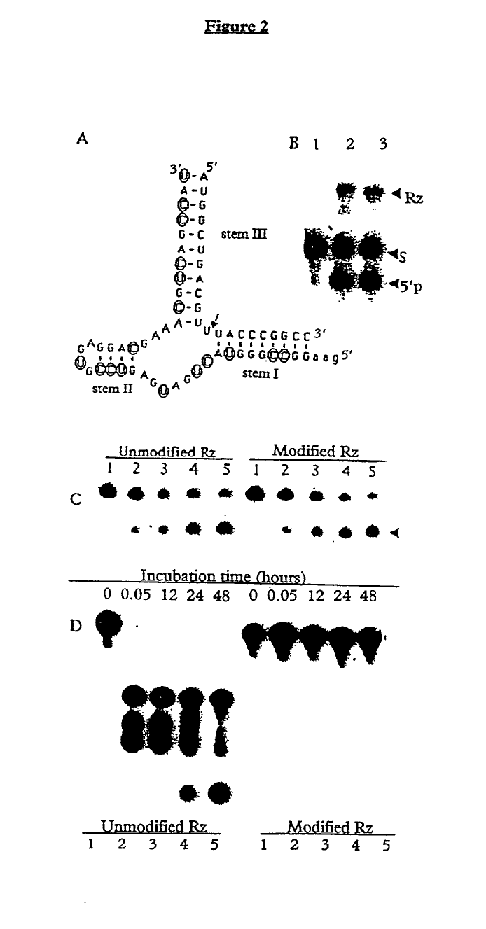

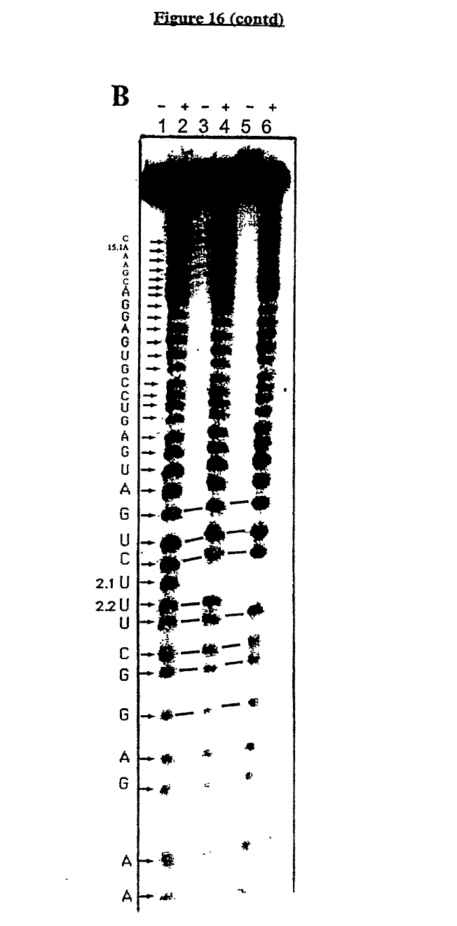

In the case of a hammerhead type ribozyme, the ribozyme comprises three stems (helices I, II and III, see for example FIG. 1A and FIG. 15), connected by single-stranded regions that contain conserved bases that are required for ribozyme cleavage activity. The specificity of the ribozyme is defined by the base composition of helix I and III (recognition sequences). For such hammerhead ribozymes, it is preferred that less than 30% (to as few as none) of the ribonucleotides in helix I are pyrimidines (e.g. 30%, 20% or 10% or less of the ribonucleotides in helix 1 are pyrimidines). Thus, for example, depending on the number of bases in helix I, 3 or less pyrimidines (i.e. 3, 2, 1 or 0 pyrimidines in helix I) may be present. It is furthermore especially preferred in hammerhead ribozymes that bases 2.1, 2.2 and 15.2 are purine bases (according to the numbering in FIG. 1A and FIG. 15). Even more preferably, bases 2.1 and 2.2 are purine bases and most preferably, base 2.1 is a purine base.

These principles may be used as the basis for the design of modified ribozymes according to the invention which are stable and have sustained cleavage activity. In particular, the invention surprisingly and unexpectedly permits uniform modification of ribozymes (i.e. uniform replacement of all pyrimidines with their 2′-amino modified analogues) whilst retaining cleavage activity (i.e. catalytic activity)

Especially preferably at least 80%, e.g. at least 90% catalytic activity is achieved (as compared to the corresponding unmodified (i.e. underivatised atthe ribozyme 2′position). Indeed, improvements in catalytic activity (relative to the corresponding unmodified ribozyme) may be achieved and are included within the scope of the invention.

Particularly preferably, the ribozyme which is modified is 2′-amino modified at at least positions 4 and 7 according to the numbering given in FIG. 1A and FIG. 15. For example, in a preferred embodiment, a hammerhead ribozyme is provided which has less than 50% pyrimidine nucleotides, wherein 90% of these bases are 2′-amino modified (including positions 4 and 7) and which exhibit improved stability and more than 90% of the catalytic activity of the unmodified ribozyme.

Modified ribozymes of the invention are capable of cleaving mRNA or pre-mRNA (ie. the RNA transcript prior to splicing to remove introns) for which they have specificity. It will however be appreciated that providing the target oligonucleotide satisfies the specificity requirements of the ribozyme, ribozymes of the invention may be used for their cleavage. Thus the ribozymes may be used to cleave DNA/RNA or RNA/PNA hybrids etc.

As indicated above, a ribozyme comprises a conserved central portion flanked by binding site recognition sequences. The conserved central region may for example be the known hammerhead or hairpin motifs, or an operable portion or analog thereof. This central portion conveniently has the hammerhead motif 5′ CUGANGA(N)ₓNNNN(N′)ₓGAAA 3′, wherein N represents A, C, G or U; x is 2, 3, 4 or 5; and N′ represents a ribonucleotide, ie. A, C, G or U, such that (N′)ₓ is complementary to (N)ₓ to allow the formation of Watson-Crick hydrogen bonding. This corresponds to loop 2 (helix II) of the hammerhead ribozyme. For example the sequence may be 5′ CUGAUGAGUCCGUGAGGACGAAA 3′ in which the respective bases are numbered: 3, 4, 5, 6, 7, 8, 9, 10.1, 10.2, 10.3, 10.4, L2.1, L2.2, L2.3, L2.4, 11.4, 11.3, 11.2, 11.2, 12, 13, 14, 15.1.

The flanking sequences are generally in the order of 5 to 15, preferably 6 to 10, especially preferably e.g. 7 or 8 nucleotides in length, and are selected to be complementary to a region of the target RNA adjacent to its RUH (R=purine, U=uracil, H=A, C, T) triplet in the RNA which is cleaved. In designing ribozymes for particular target RNA, absolute complementarity of the flanking region is not required although at least 80% homology or sequence identity or more should be obtained over an at least 5 nucleotide base region to provide the binding specificity (sequence homologies or sequence identities may be calculated as defined as specified below). In some cases imprecise (i.e. less than absolute) complementarity may be advantageous when the ribozyme is to be used for targeting mRNA of e.g. different species which have slightly different sequences. Thus, the ribozyme is preferably designed with reference to regions on the target mRNA which are common or highly conserved between species.

Preferably the overall sequence of the ribozyme will be from 30 to 45, especially 35 to 40, for example 37 or 38 bases in length, particularly with flanking sequences each of 7 or 8 bases.

Ribozymes according to the invention may have sequences of known ribozymes, may be truncated or derivatized ribozymes or may be novel ribozymes designed in accordance with the above. In each case, the ribozymes are 2-NH₂ modified on the pyrimidine bases as described herein. Ribozymes of the invention include those which have been further derivatized (except at the 2′ position of the pyrimidine bases), e.g. by the use of phosphorothioate links at the terminal 3′ end to improve stability. Additional protection against RNAse cleavage may also be achieved by introducing an inverted T(iT) at the 3′ end of the ribozyme. Techniques for achieving this are well known in the art.

The preparation of different ribozymes is well known in the art and widely described in the literature, see e.g. Uhlenbøck, 1987, Nature, 328, p596-600; Forster et al., 1987, Cell, 50, p9-16; Pley et al., 1994, Nature, 372, p68-74, Tuschl et al., 1994, Science, 266, p785-789; U.S. Pat. Nos. 5,496,698; 5,144,019; 5,272,262 and Hampel et al., 1989, Biochemistry, 28, p4929; Hampel et al., 1990, Nucl. Acids Res., 18, p299 and U.S. Pat. No. 5,527,895.

In general, ribozymes may be produced by chemical synthesis or by transcription or by a combination thereof. Alternatively, To obtain the modified ribozymes of the invention, derivatized bases may be used during the synthesis/transcription step as described by Aurup et al., 1992, Biochemistry, 31, p9636-9641. These bases may additionally be derivatized in other ways, or may be derivatized after generation of the modified ribozymes. If transcription is to be used, the DNA from which transcription is to be performed may be appropriately amplified (e.g. by PCR) and/or may be inserted into a vector, e.g. a plasmid, which may optionally be transfected into a host cell.

As will be seen from the above, various ribozymes may be produced and modified according to the invention. Preferred ribozymes for modification according to the invention include:

| | |
|---|---|
| 5′-GAAGGCCGGGUACUGAUGAGUCCGUGAGGACGAAACGUCAGCCAU-3′ | [SEQ ID NO: 1] |
| (PKCα ribozyme); | |
| 5′-GAAGAGGCUGACUGAUGAGUCCGUGAGGACGAAACAUAGGCACCG-3′ | [SEQ ID NO: 2] |
| (TNFα ribozyme); | |
| 5′-GGGAAGGCCGGGAACUGAUGAGUCCGUAGGACGAAACGUCAGCCAU-3′ | |
| 5-GGGGGAACUGAUGAGUCCGUGAGGACGAAACGUCAGCCAU-3′ | [SEQ ID NO: 3] |
| (human PKCα ribozymes); | |
| 5′-GGAAAGACUGAUGAGUCCGUGAGGACGAAAGCAGAAAGUGCAUGG-3′ | [SEQ ID NO: 4] |
| (rat vascular endothelial growth factor ribozyme); and | |
| 5′-GAGCAGACUGAUGAGUCCGUGAGGACGAAAGUUCAUGG-3′ | [SEQ ID NO: 5] |
| (human vascular endothelial growth factor ribozyme) | |

and sequences which have at least 70 or 80% or more, preferably at least 85 or 90% homology or sequence identity thereto or which would hybridise to a complement of said sequences under conditions of high stringency, or functionally equivalent analogs, variants or fragments thereof.

Modified ribozymes having any specificity are included within the scope of the invention. However, preferred ribozymes include those having specificity for RNA sequences which are involved in the PKCα and Bclx_{L} signalling pathway identified and referred to in Example 5 of this application. Particularly preferred are ribozymes having specificity for the PKC enzymes, preferably the PKCα enzyme and most preferably the PKCα target site common to both human and rat having the following sequence:

5′GGGGGGGACCAUGGCUGACGUUU3′; [SEQ ID. NO: 6]

Other preferred ribozymes according to the invention may be directed against the target site AU as indicated in SEQ ID NO: 6 above. Such ribozymes preferably have a sequence selected from one of the following:

| | |
|---|---|
| 5′GTCAGCCAGGCTAGCTACAACGAGGTCCCC-3′ | [SEQ ID |
| | NO: 7] |
| 5′GTCAGCCAGGCTAGCTACAACGAGGTCCCCCC-3′ | [SEQ ID |
| | NO: 8] |
| and | |
| 5′AAACGTCAGCCAGGCTAGCTACAACGAGGTCCCCCC-3′ | [SEQ ID |
| | NO: 9] |

and sequences which have at least 70 or 80% or more, preferably at least 85 or 90% homology or sequence identity thereto or which would hybridise to a complement of said sequences under conditions of high stringency, or functionally equivalent analogs, variants or fragments thereof.

These ribozymes (SEQ ID NOS: 7, 8 and 9) are examples of short PKCα DNA ribozymes. These ribozymes cleave a RNA phosphoester located between an unpaired purine and paired pyrimidine residue (Stephen et al., 1997, PNAS 94: 4262-4266). They have been found to cleave the substrate and to block cell growth. Many other modifications suitable for antisense DNA which are known in the art (e.g. phosphorothioate analogues) may be advantageous to stabilise these above-mentioned ribozymes.

As referred to herein, sequence identity or sequence homology may be determined using the Fasta search (Pearson and Lipman (1988), Proc. Natl. Acad. Sci. USA 5: 2444-2448) as part of the GCG packages using default values; word size: 6; Gap creation penalty: 12.0; Gap extension penalty: 4.0, and constant Pam factor.

Conditions of high stringency may readily be determined according to techniques well known in the art, as described for example in Sambrook et al., 1989, Molecular Cloning, A Laboratory Manual, 2nd Edition. Hybridising sequences included within the scope of the invention are those binding under non-stringent conditions (6×SSC/50% formamide at room temperature) and washed under conditions of high stringency (e.g. 2×SSC, 65° C.), where SSC=0.15 M NaCl, 0.015M sodium citrate, pH 7.2.

Functionally equivalent analogs, variants or fragments, are those which have been modified and/or truncated but which retain the functional activity, i.e the catalytic activity of the ribozyme. This includes both sequence modification in the sense of nucleotide substitution, addition or deletion (whether of single or multiple contiguous or non-contiguous nucleotides) and also chemical derivatives of nucleotide residues or groups. In particular this includes molecules which have been derivatized as described herein at sites other than the 2′ positions of the pyrimidine bases.

Preferably 90% or more of the above pyrimidine bases are 2′-amino derivatized, especially preferably global uniform 2′-amino derivatization is performed.

Thus, viewed from a preferred aspect, the present invention provides ribozymes with the sequences defined above and their functionally equivalent analogs, variants and fragments, wherein 90% or more of the pyrimidine nucleotides are modified at the 2′-position, wherein said pyrimidine nucleotides are modified to 2′-amino pyrimidine nucleotides and said ribozyme exhibits improved stability to RNAse degradation and exhibits 20% or more, for example 50% or more, catalytic activity of the unmodified ribozyme.

As mentioned previously, ribozymes according to the invention have a number of different applications for cleaving target RNA either in vitro or in vivo. For example, in vitro, ribozymes of the invention may be used to cleave specific RNA in RNA samples or cell lysates or to alter gene expression of cells or explants in culture.

For in vivo applications, ribozymes may be administered systemically or locally for, for example, research, therapeutic or cosmetic purposes. It will also be appreciated that the ribozymes inherently carry antisense capabilities in addition to their catalytic properties which can additionally serve to prevent gene expression.

Thus viewed from a further aspect, the present invention provides the use of modified ribozymes of the invention in hydrolysing RNA in vitro or in vivo, or as antisense molecules.

Modified ribozymes of the invention may be used to reduce or suppress the expression of proteins correlating to the mRNA which is targeted and thus are particularly suitable for treating or preventing any condition which may be corrected or improved by altering (e.g. suppressing or eliminating) gene expression of one or more gene products by cleaving partially or completely the RNA transcribed from said gene.

Thus, viewed from a further aspect, the present invention provides a method of treating or preventing a disease or condition by administration of one or more ribozymes of the invention. Alternatively viewed, the present invention provides ribozymes of the invention for use as a medicament, i.e. for use in therapy.

More particularly, these aspects of the invention provide the use of a ribozyme of the invention in the manufacture of a medicament for treating or preventing a disease or condition responsive to an alteration in the expression of a gene wherein said ribozyme is capable of cleaving the RNA transcribed from said gene.

Also provided is a method of treating or preventing a disease or condition responsive to an alteration in the expression of a gene, said method comprising administering a ribozyme of the invention wherein said ribozyme is capable of cleaving the RNA transcribed from said gene.

The ribozymes may be administered parenterally, e.g. subcutaneously, intravenously, intraarterially, and intramuscularly, either by injection or infusion. Alternatively, prolonged release formulations may be administered, e.g. by subcutaneous depot dosaging. Preferably however the ribozymes will be administered by injection or infusion directly into the vasculature of the patient.

Ribozyme delivery techniques and methods are known in the art are described in the literature and any of such known or described procedures or carriers or vehicles may be used, e.g. liposome vehicles as described in Sioud et al. in J. Mol. Biol. 223: 7303-7307, 1992 and J. Mol. Biol. 222: 619-629, 1994.

Whilst the invention provides catalytically active ribozymes with improved stability over those known in the prior art, prior art methods of administering less stable ribozymes (either in vitro or in vivo) may also be used. Thus, for example, ribozymes may be delivered directly to relevant sites by microinjection or transfection, e.g. in the form of CaCl₂ or cationic liposomes. Alternatively the ribozymes may be derivatized to allow passage across the cell membrane, e.g. by the addition of appropriate lipophilic groups.

The dosage used will depend on the condition being treated, and the age, gender, size and species of the patient. Typically however doses to humans may be expected to be 0.1 to 20 mg/kg bodyweight, preferably 1 to 10 mg/kg, more preferably 1 to 5 mg/kg, administered one to four times daily.

In particular, it has been found that ribozymes of the invention may be used to prevent or reduce proliferation of rapidly dividing cells. Clearly this has applications in the treatment of tumours such as cancers. Indeed it has been found that a protein kinase Cα specific ribozyme (PKCα Rz) and a vascular epithelial growth factor ribozyme (VEGF Rz) blocked malignant glioma growth in vitro and in vivo.

Thus, from a yet further aspect, the present invention provides a method of inhibiting cell proliferation, wherein said method comprises the administration of one or more modified ribozymes of the invention to said cells.

In particular a method of treating cancer in a patient is provided wherein said method comprises administration of ribozymes of the invention to said patient. As used herein, the term “cancer” includes any neoplastic, malignant or pre-malignant condition, including cancer of any of the tissues or cells of the body. Thus, not only solid tumours are covered, but any cancer of the haemopoeitic system, as well as metastases etc. Preferably however cancers covered by the present invention comprise malignant or anaplastic proliferations of cells. Especially preferred is the administration of at least one of:

| | |
|---|---|
| 5′-GAAGGCCGGGUACUGAUGAGUCCGUGAGGACgAAACGUCAGCCAU-3′ | [SEQ ID NO: 1] |
| (PKCα ribozyme); | |
| 5′-GGGAAGGCCGGGAACUGAUGAGUCCGUGAGGACGAAACGUCAGCCAU-3′ | |
| 5-GGGGGAACUGAUGAGUCCGUGAGGACGAAACGUCAGCCAU-3′ | [SEQ ID NO: 3] |
| (human PKCα ribozymes); | |
| 5′-GGAAAGACUGAUGAGUCCGUGAGGACGAAAGCAGAAAGUGCAUGG-3′ | [SEQ ID NO: 4] |
| (rat vascular endothelial growth factor ribozyme); | |
| 5′-GAGCAGACUGAUGAGUCCGUGAGGACGAAAGUUCAUGG-3′ | [SEQ ID NO: 5] |
| (human vascular endothelial growth factor ribozyme), | |
| 5′-GTCAGCCAGGCTAGCTACAACGAGGTCCCC-3′ | [SEQ ID NO: 7] |
| 5′-GTCAGCCAGGCTAGCTACAACGAGGTCCCCCC-3′ | [SEQ ID NO: 8] |
| and | |
| 5′-AAACGTCAGCCAGGCTAGCTACAACGAGGTCCCCCC-3′ | [SEQ ID NO: 9] |

or sequences which have at least 70 or 80% or more, preferably at least 85 or 90% homology or sequence identity thereto or which would hybridise to a complement of said sequences under conditions of high stringency, or functionally equivalent analogs, variants or fragments thereof, which have been 2′-amino modified as described above.

It will be appreciated from the Examples herein that ribozymes without catalytic activity also have the above described anti-proliferative effect. Thus, from a further aspect, the invention provides a method of inhibiting cell proliferation using modified ribozymes as described above, but which may not retain the stated catalytic activity, e.g. they may be mutated in the catalytic region. This aspect also extends to unmodified ribozymes having the above described sequences. Ribozymes of the invention which are catalytically inactive or exhibit reduced catalytic activity e.g. as compared with the native or unmodified ribozyme are particularly useful in the methods of treatment, in vitro methods and uses according to the invention as described herein.

Furthermore, the novel ribozymes described herein, their homologous or complementary sequences or their functionally equivalent analogs, variants or fragments and their uses form further aspects of the invention.

Pharmaceutical compositions comprising modified ribozymes of the invention form a further aspect of the invention. Pharmaceutical compositions may be formulated in combination with other active agents e.g. other therapeutic agents or drugs in accordance with formulation techniques known in the art. For example, pharmaceutical compositions may include the ribozymes according to the invention and/or other active agents such as therapeutic agents or drugs which interfere with the PKCα signal pathway as identified and discussed in Example 5 of this application. Such drugs may for example cause or enhance apoptosis and are useful e.g. in cancer therapy.

Further medical uses for the ribozymes of the invention, in particular ribozymes against PKCα and/or VEGF, include treating patients with autoimmune diseases. VEGF is involved in the formation of the pannus seen in patients with rheumatoid arthritis. The ribozymes may, in this regard, be locally injected into a patient's joints.

The novel “unmodified” (in the sense of underivatised at the 2′-OH position) ribozymes may be delivered to patients in any known manner. Thus not only is delivery of exogenous or “pre-formed” ribozymes as described above encompassed, but also the ribozymes may be generated in situ (i.e. “endogenous” ribozyme delivery) by expression of administered or delivered coding sequences e.g. in the form of an expression vector e.g. a plasmid or a viral vector. Techniques for this are known in the art (see e.g. Feng et al., Nat. Biotechnol. 15: 866-870, 1997).

As may be seen from Example 6 of the application, cleavage activity of the ribozymes of the invention may be affected by the presence or absence of various metal ions, particularly thiophilic ions such as Mn²⁺, Mg²⁺, Zn²⁺ and Co²⁺ ions. Thus, the ribozymes of the invention, modified or unmodified may be used in conjunction with such thiophilic ions in order to enhance catalytic (cleavage) activity of the ribozymes. Preferably, then, the ribozymes of the invention may be used in the in-vitro method, uses and methods of treatment as described herein in combination with or in conjunction with a metal ion selected from Mn²⁺, Mg²⁺, Zn²⁺ and Co²⁺ ions, most preferably Mn²⁺. The metal ions in this aspect of the invention may be used in separate, sequential, or combined preparations for administration to a patient or for in vitro use. If necessary or desired, such metal ions may alternatively be excluded from any composition containing the ribozymes of the invention in order to enhance or suppress catalytic activity thereof.

The following Examples are given by way of illustration only, with reference to the following figures:

FIG. 1A shows a hammerhead mouse TNF-α ribozyme with bound RNA substrate illustrating the numbering system used herein. The cleavage site is indicated by an arrow. The 2′-amino pyrimidine nucleotides which are modified are circled;

FIG. 1B shows the multiple turnover reaction kinetics of the unmodified and 2′-amino modified mouse TNF-α ribozyme at time points 15, 30, 60 and 90 minutes (lanes 2-5, respectively) analysed on a 15% gel visualized by autoradiography. Rz=ribozyme, s=substrate and 5′P=5′-cleavage product;

FIG. 1C shows the results of the stability of TNF-α ribozyme in 10% FCS in which time point 0 was taken prior to serum addition;

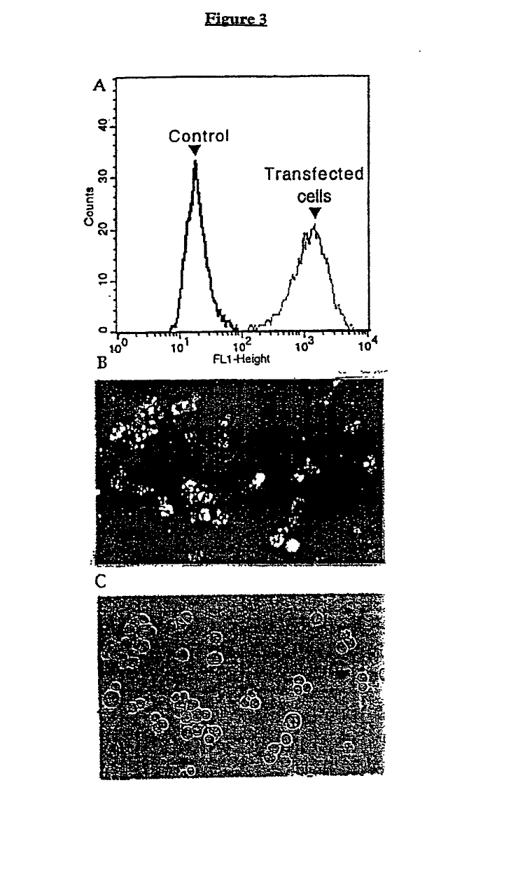

FIGS. 2A, C and D correspond respectively to FIGS. 1A, B and C using PKCα Rz. FIG. 2B shows in vitro cleavage of a 21 nt substrate by the unmodified (lane 2) and 2′-amino pyrimidine modified PKCα Rz (lane 3) for 60 minutes under the conditions of FIG. 1B, visualized by PhosphorImager;

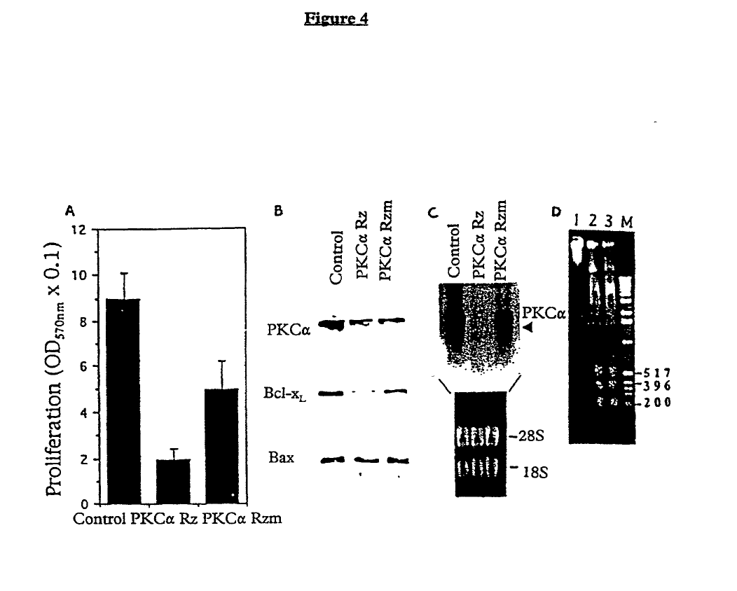

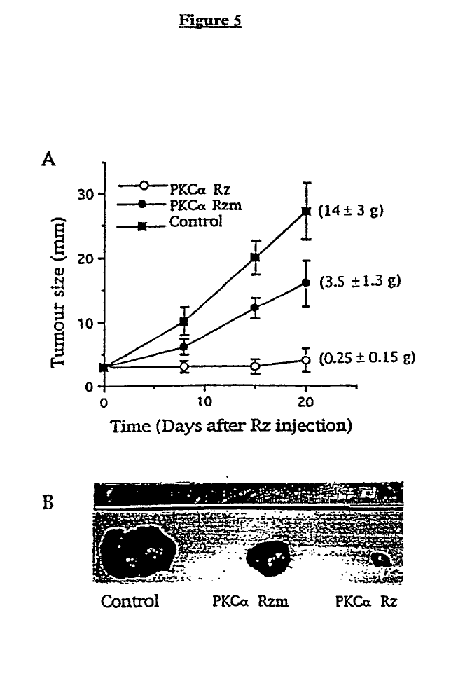

FIGS. 3A, B and C show ribozyme uptake by BT4Cn glioma cells after transfection as assessed by (A) flow cytometry, (B) fluorescence or (C) light microscope. The data is representative of 5 experiments;

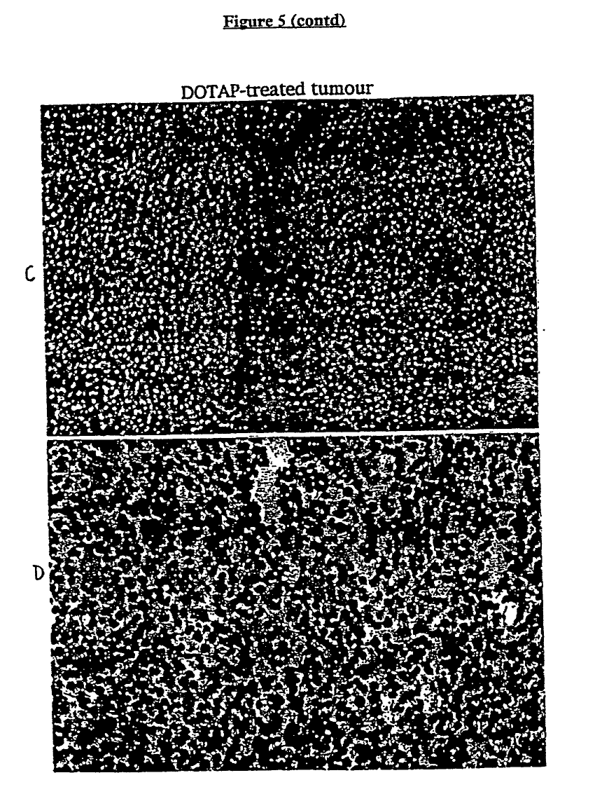

FIG. 4 shows the effect of PKCα Rz and Rzm on cell proliferation, (A) shows the effect of PKCα Rz on proliferation of BT4Cn cells as assessed by the MTT assay after transfection for 42 hours, (B) shows a Western blot of cytoplasmic proteins (15 μg) prepared after 42 hours of transfection with PKCα Rz or PKCα Rzm, probed with anti-PKCα, Bcl-XL or Bax, (C) shows a Northern blot of total RNA extracted after 42 hours of transfection, separated on a 1% agarose gel, transferred to a nylon membrane and probed with a ³²P-antisense probe specific for the PKCα. Ribosomal RNAs served as an internal control for RNA loading and (D) shows analysis of crude DNA preparations after 36 hours transfection time and lysis of cells, on 1% agarose gel stained with ethidium bromide, lane 1=control (transfected with DOTAP), lane 2 and 3=cells transfected with PKCα Rz; M=1 kb DNA ladder;

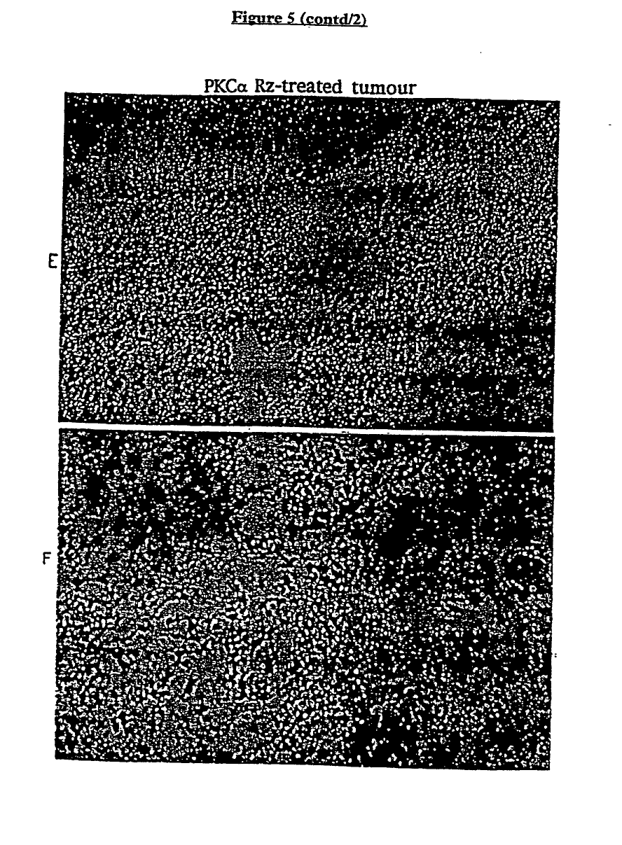

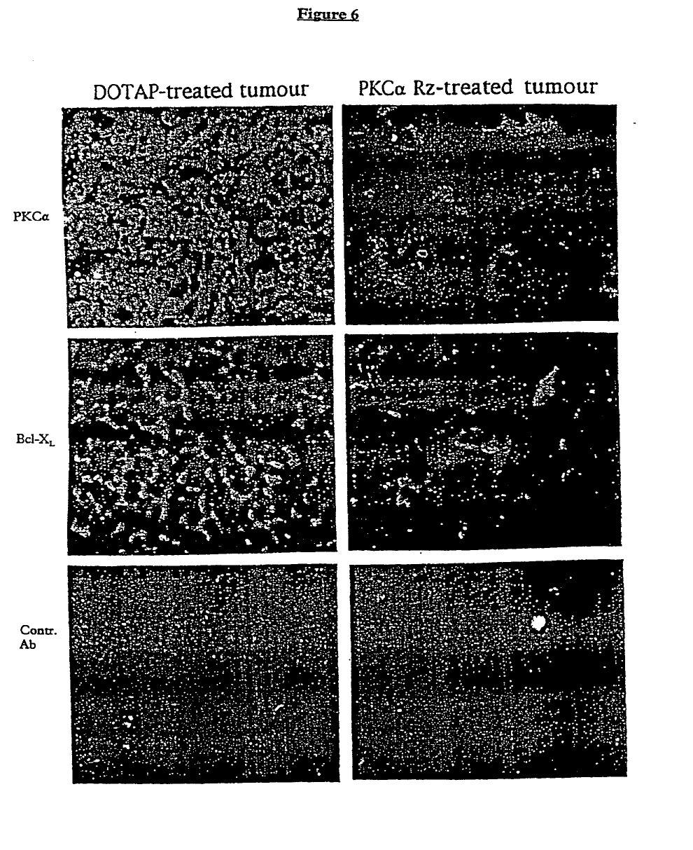

FIG. 5 shows the effect of modified PKCα on tumour growth in vivo, in respect of (A) tumour size, graphically, (B) tumour size (visually). FIGS. 5C to F show the histology of 5 mm cryostat section from a DOTAP-treated tumour (C, D) or from a PKC Rz-treated tumour (E, F). D and F are as C and E, respectively, but at higher magnification;

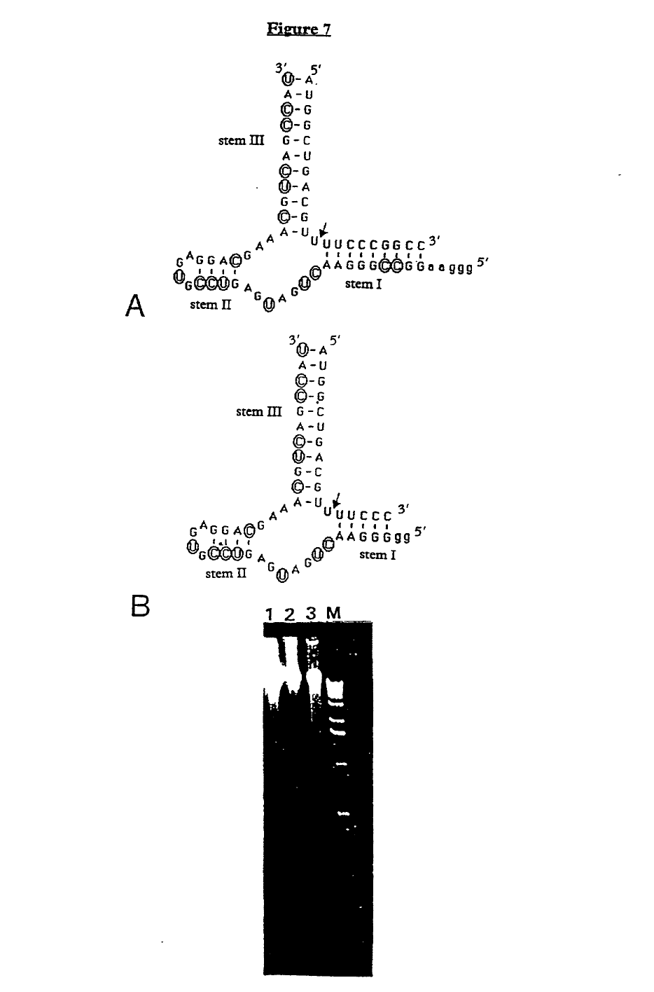

FIG. 6 shows PKCα and Bcl-Xl gene expression in DOTAP and in PKCα-treated tumours stained with anti-PKCα, Bcl-XL or with normal rabbit IgG as control and revealed by a FITC-conjugated goat anti-rabbit IgG;

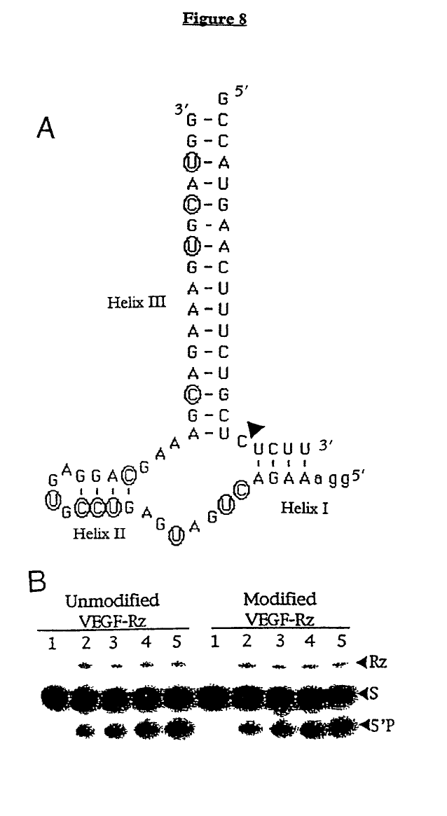

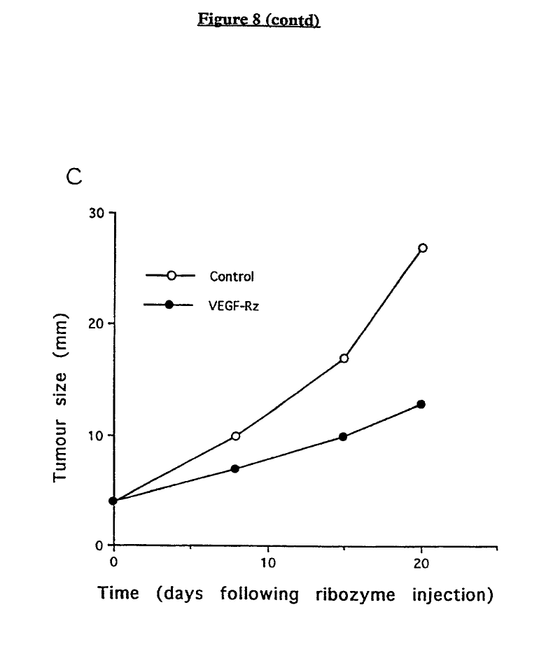

FIG. 7A shows the binding of human PKCα ribozymes to their RNA substrates. The cleavage sites are indicated by arrows. The 2′-amino pyrimidine nucleotides which are modified are circled; FIG. 7B shows the effect of the lower ribozyme on human glioma cell survival as assayed by DNA degradation; and

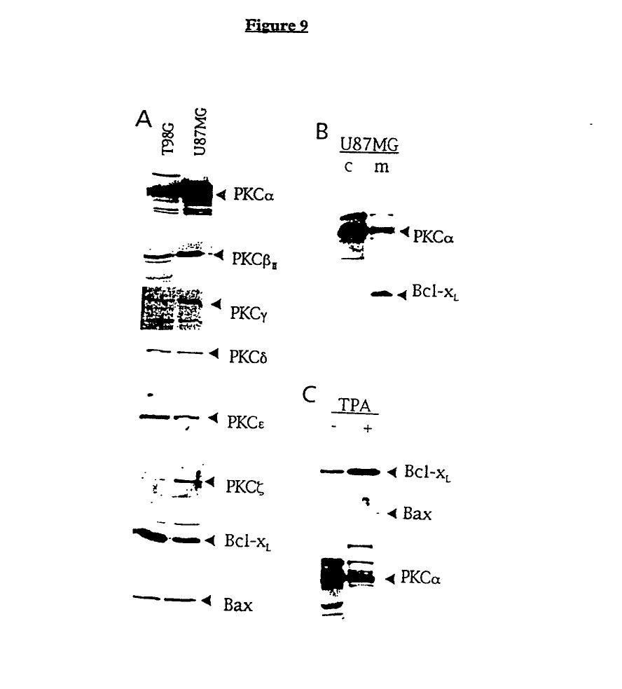

FIG. 8A shows the binding of a vascular endothelial growth factor ribozyme to its substrate; FIG. 8B shows the results of multiple turnover reactions of unmodified modified VEGF ribozymes as in FIG. 1B, visualized by PhosphorImager at time pints 5, 10, 15 and 30 Minutes (lanes 2-5, respectively); FIG. 8C shows the effect of this ribozyme on tumour growth as in FIG. 5A.

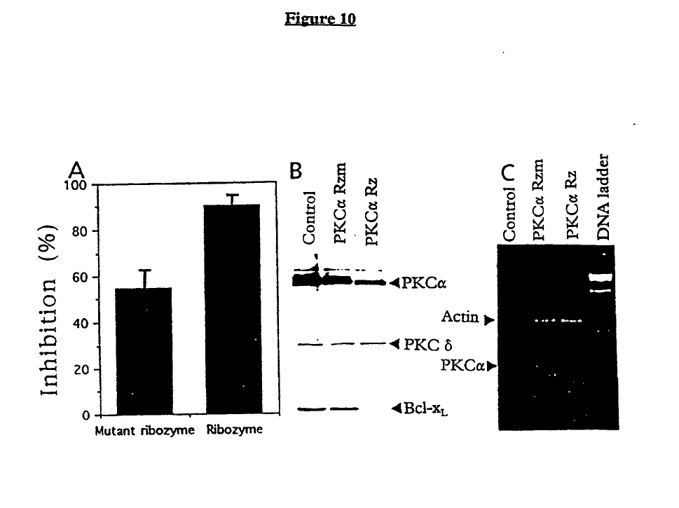

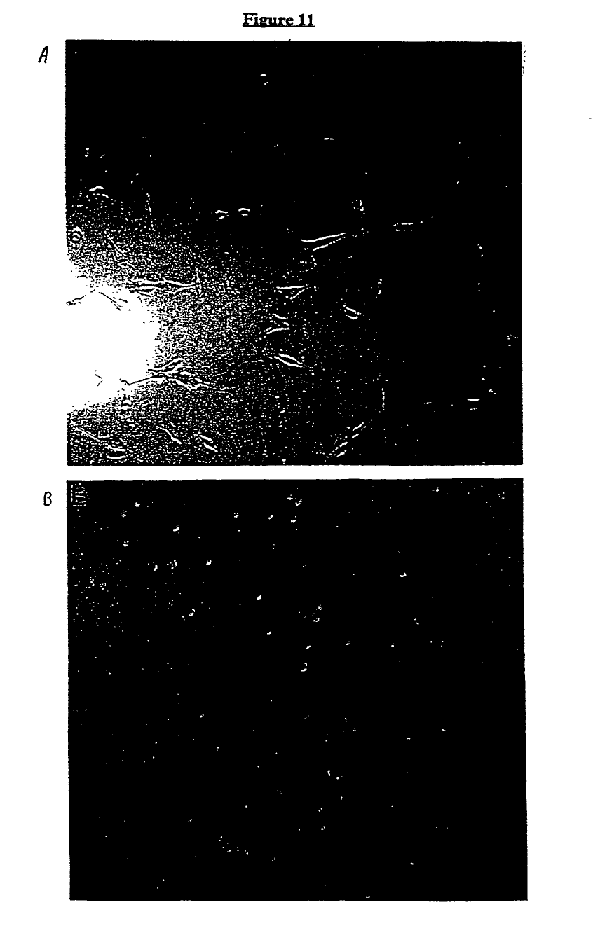

FIG. 9 shows Western blot analysis. 9A) Expression of the PKC isoforms, the Bcl-x_{L}, the Bax proteins by T98G and U87MG human glioblastoma cell lines. Both glioma cell lines overexpress the PKCα and Bcl-x_{L} proteins. 9B) Analysis of PKCα and Bcl-x_{L} in the cytosol and the membrane fractions prepared from U87MG glioma cells. m=membrane fraction, c=cytosol fraction. Similar results were obtained with T98G cells. 9C) Upregulation of Bcl -x_{L} gene expression by TPA. Cells were incubated with TPA for 5 hours. Protein extracts from unstimulated and stimulated cells were prepared and 15 μg from each sample were analysed by immunoblotting using Bcl-x_{L}, Bax or PKCα antibodies. Bands that represent the investigated proteins are indicate by arrows. Each data is representative of 4 independent experiments.

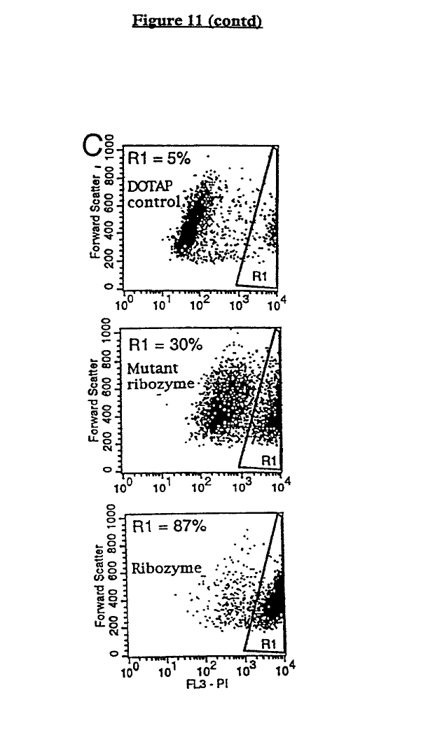

FIG. 10 shows inhibition of glioma cell growth and PKCα gene expression by the ribozyme. 10A) Inhibition of cell proliferation. Cells were transfected for 48 hours and cell proliferation was measured by the MTT assay. Inhibition was expressed as a percentage of the DOTAP-treated cells. 10B) The ribozyme reduced the expression of the PKCα and Bcl-x_{L}, but not PKCδ. After 48 hours transfection time, protein extracts were prepared from DOTAP-(control), mutant ribozyme- and ribozyme-treated cells and 15 μg from each sample were analysed by Western blot using specific antibodies 10C). The ribozyme eliminated its target mRNA in the cell. Total RNA was prepared and the expression of PKCα was detected by RT-PCR as described in Example 5, Materials and Methods. Each data is representative of least of 4 independent experiments.

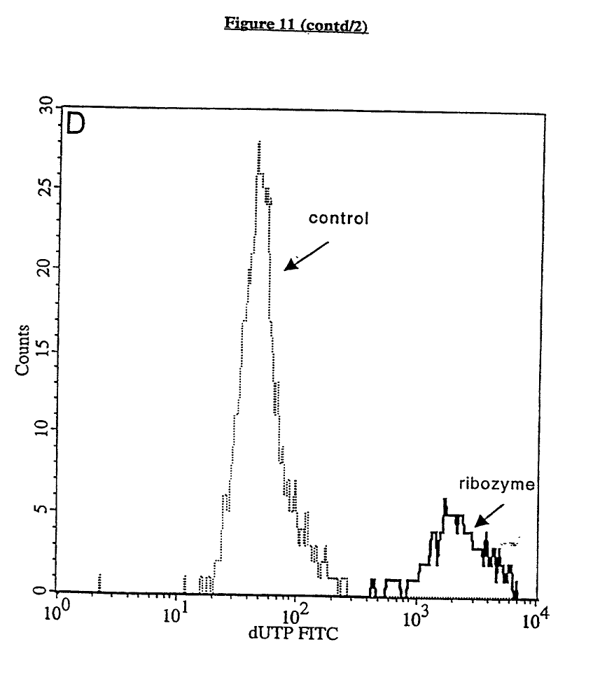

FIG. 11 shows that ribozyme inhibition of the PKCα gene expression induces apoptosis in glioma cells. Light microscope image of PKCα ribozyme treated U87MG cells. DOTAP- (11A) and ribozyme-treated cells (11B) for 48 hours were photographed to illustrate the morphological changes produced by the inhibition of the PKCα isoform. 11C) Cell death in glioma cells as detected by propidium iodide (PI) positive cells. DOTAP-, mutant ribozyme- and ribozyme-treated cells for 24 hours were stained with PI and analysed by flow cytometry. 11D) Quantification of cellular DNA fragmentation by the TUNEL method. DOTAP- and ribozyme treated-cells for 48 hours were analysed by the TUNEL method as described in Example 5, Materials and Methods. Each data is representative of 4 independent experiments.

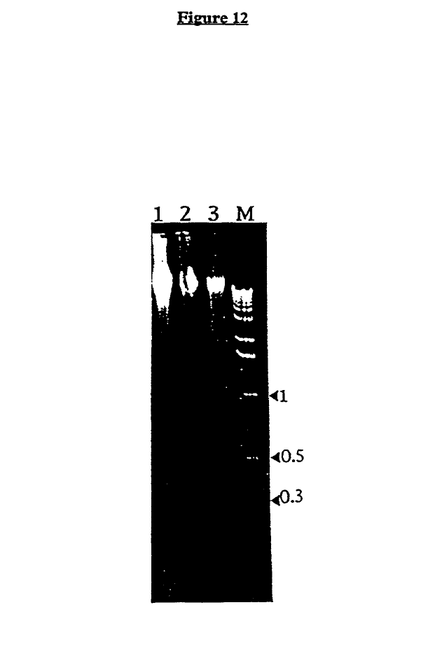

FIG. 12 shows induced DNA fragmentation by the ribozyme. Following 24 hours transfection time, cells were lysed, DNA crude preparations were prepared, analysed by a 1% agarose gel and then stained with ethidium bromide. Lane 1, DOTAP-treated cells; Lane 2, mutant ribozyme-treated cells; and Lane 3, ribozyme-treated cells. M, 1 Kb DNA ladder. A “DNA ladder” is evident in ribozyme-treated cells. Data is representative of 4 independent experiments.

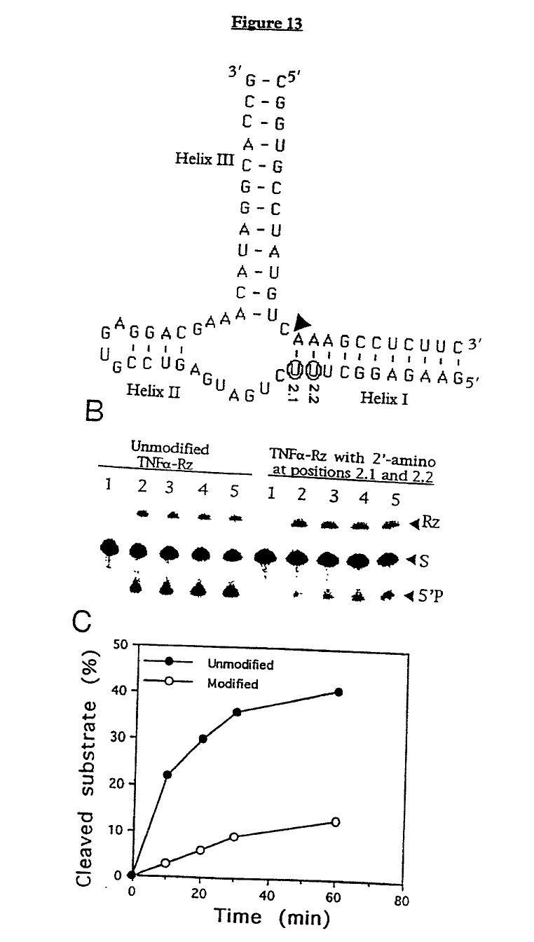

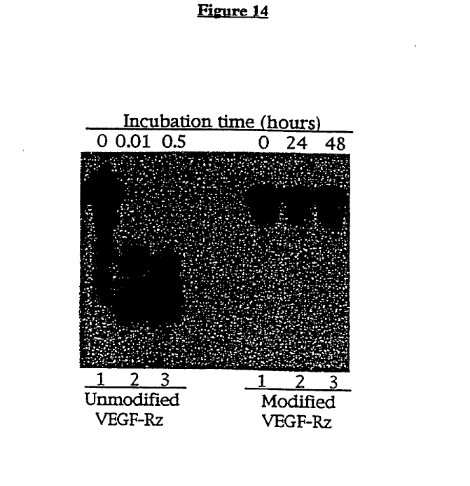

FIG. 13 shows in vitro cleavage activity of the TNFα with 2′-amino-uridine at position 2.1 and 2.2. (A) Base-pairing of the ribozyme with its RNA target site. The cleavage site is indicated by an arrow. The 2′-amino uridines are circled. (B) An example of multiple turnover reactions of unmodified and modified ribozymes. Cleavage reactions are as in FIG. 1B. (C) Quantification of the data shown in B.

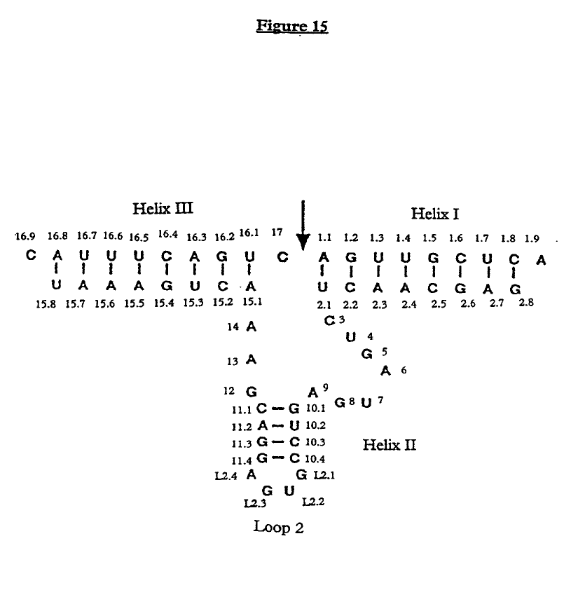

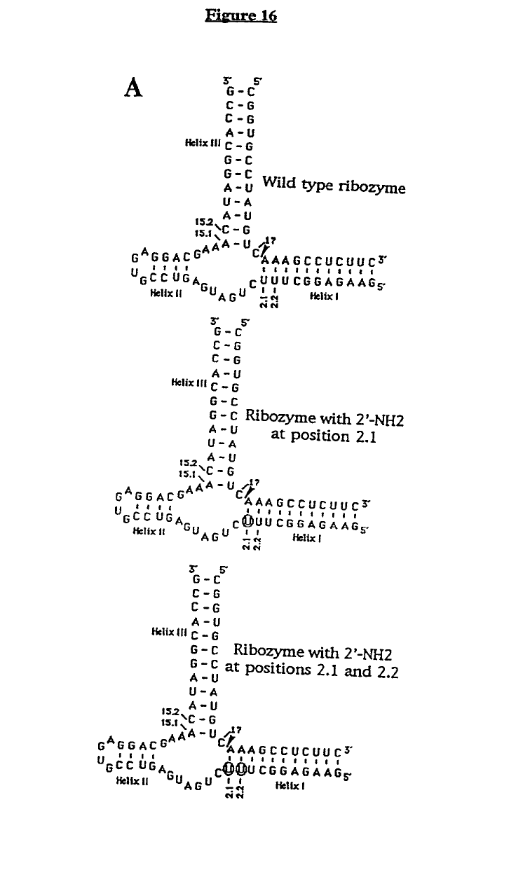

FIG. 14 shows ribozyme stability in medium containing 10% FCS. Internally labelled and PAGE purified ribozymes were incubated in RPMI supplemented with 10% FCS. At indicated times 10 μl aliquots were removed from the mixture and processed as described in Example 4, Material and Methods. Samples were analysed by 15% polyacrylamide gel with 7 M urea and analysed by PhosphoImager. Time point 0 was taken prior to serum addition.

FIG. 15 shows a representation of a typical hammerhead ribozyme and the numbering of the bases according to standard practice in the art.

FIG. 16 shows sequence and secondary structure of the TNFα ribozymes. A) Base-pairing of the TNFα ribozymes with their corresponding target RNAs. The cleavage site is indicated by an arrow. The 2′-amino pyrimidine nucleotides in the modified ribozyme are encircled. B) Autoradiography of base hydrolyzed ribozymes. Untreated (−) and base-treated ribozymes (+) were analysed by 10% denaturating Polyacrylamide gel electrophoresis. For the unmodified ribozyme this type of analysis should generate an RNA ladder (lane 2). Cleavage bands are not visible at position 2.1 (lane 4) or positions 2.1 and 2.2 (lane 6), because the hydroxyl groups required for hydrolysis are not present in the ribozyme with 2′-NH₂ at position 2.1 or in the ribozyme with 2′-NH₂ at positions 2.1 and 2.2, respectively.

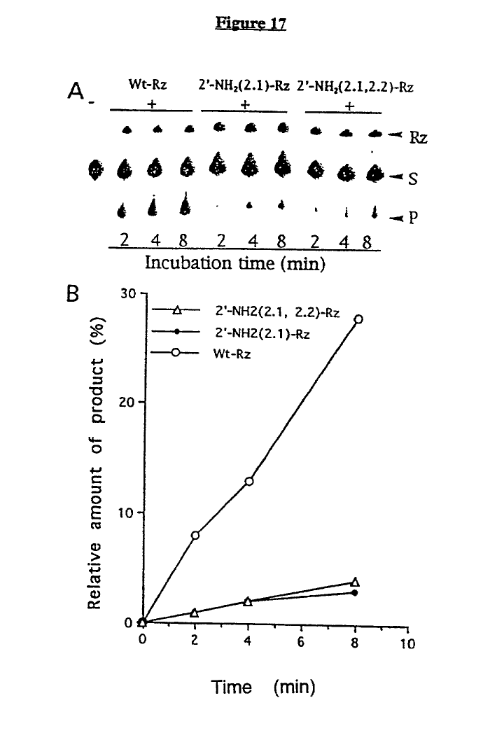

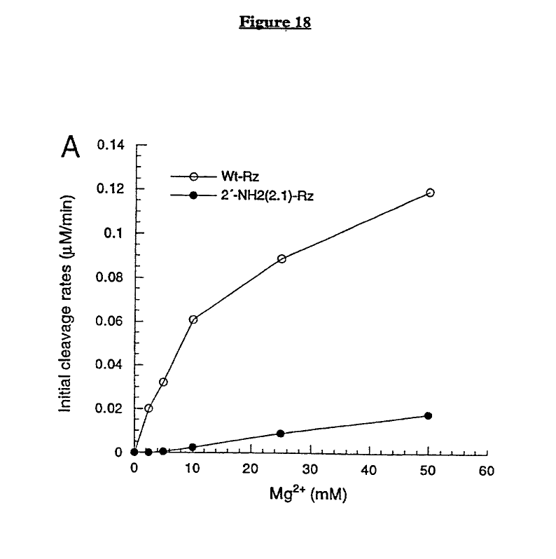

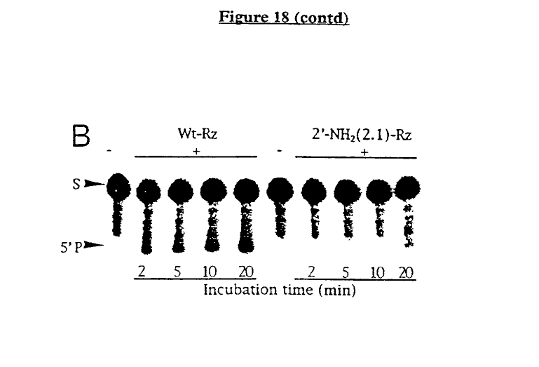

FIG. 17 shows initial time courses of the unmodified and modified ribozyme reactions. A) An example of multiple turnover reactions in the presence of Mg²⁺ ions (10 mM). 5′-labelled ribozymes (10 nM) were incubated with 5′-³²P-end-labelled target RNA (100 nM) in reaction mixtures containing 50 mM Tris HCl (pH 7.4) and 10 MgCl₂at 37° C. Aliquots were taken at the indicated time, analysed by electrophoresis on a 15% polyacrylamide gel with 7 M urea and visualised by a PhosphoImager. The 5′-cleavage product is indicated by an arrow. Wt-Rz=Unmodified ribozyme; 2′-NH₂ (2.1)-Rz=Ribozyme with 2′-amino at position 2.1; 2′-NH₂ (2.1, 2.2)-Rz=Ribozyme with 2′-amino at position 2.1 and 2.2, Rz=Ribozyme; S=Substrate and P=5′-cleavage product. B)Quantitation of the data presented in A.

FIG. 18 shows cleavage activity of the ribozymes in the presence of various Mg²⁺ concentrations. A) Initial cleavage rates of the substrate with various concentrations of Mg²⁺. Cleavage reactions contained 50 mM Tris-HCl (pH 7.4), 40 nM ribozyme and 200 nM substrate. Initial cleavage rates were obtained from the slopes of the curves for the time course reactions at the initial stage. In these experiments 4 time points were taken. B) A representative example of the cleavage in the presence of 2.5 mM Mg²⁺.

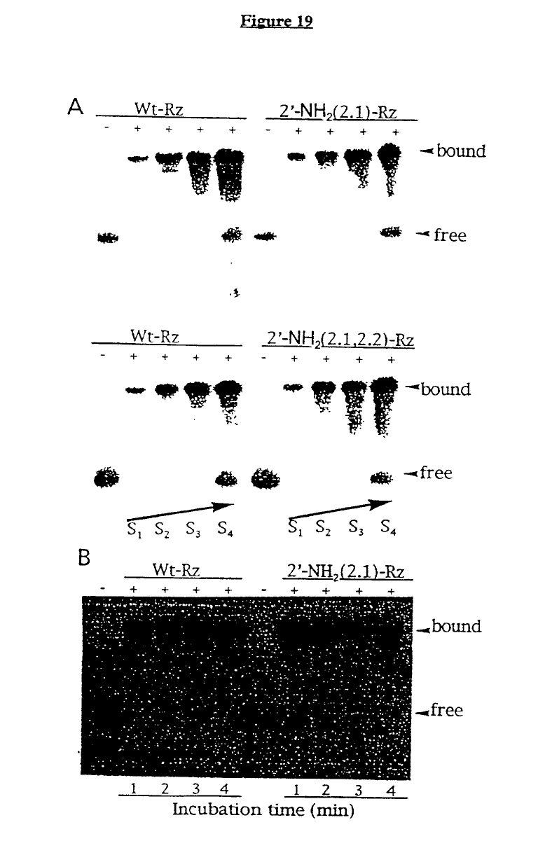

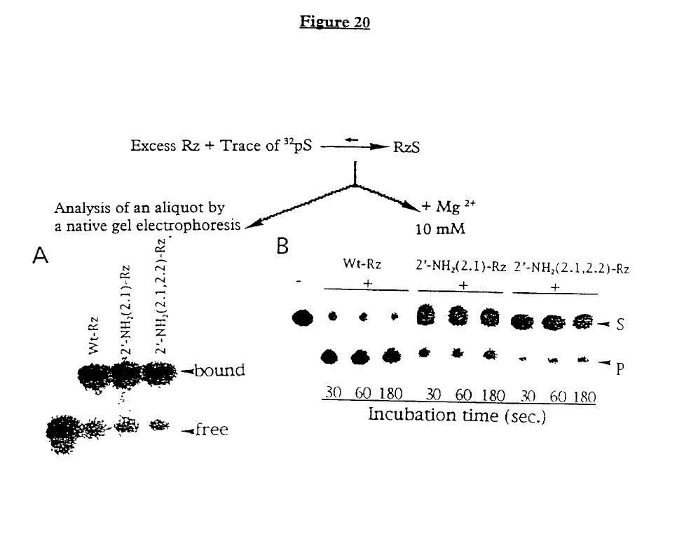

FIG. 19 shows binding of the substrate to the ribozymes. A) Binding of various concentrations of the 5′-³²P-labelled substrate to 25 nM of the unmodified or modified ribozymes. The substrate concentrations were 5, 10, 20 and 30 nM for S₁, S₂, S₃ and S₄ respectively. Samples were analysed by 15% native gel electrophoresis in TBE buffer and visualised by a PhosphoImager. B)Binding of a 5′-³²P-labelled substrate (1 nM) to the Wt-Rz (25 nM) and 2′-NH₂(2.1)-Rz (25 nM) as a function of time. Substrate and ribozymes were incubated at 37° C. in 50 mM Tris HCl (pH 7.4). Aliquots were taken at the indicated time, analysed by 15% native gel electrophoresis and visualised by PhosphoImager.

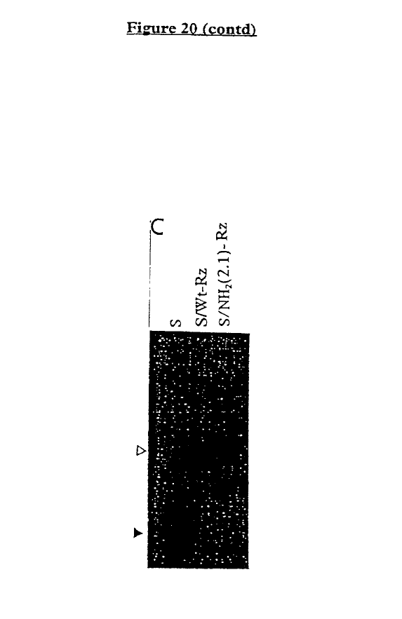

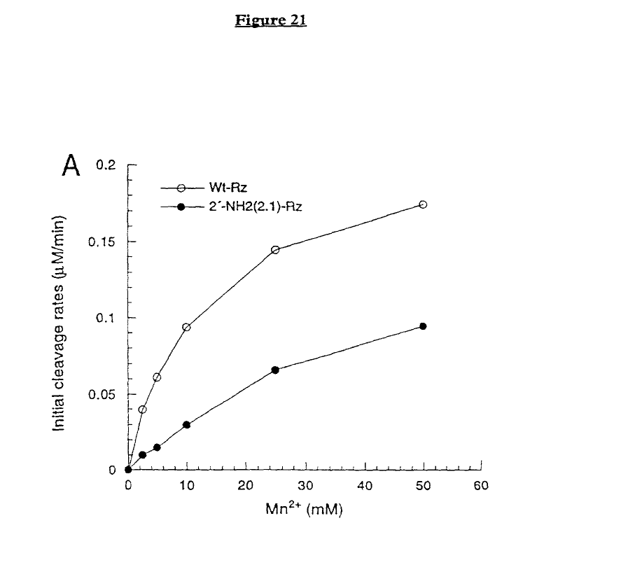

FIG. 20 shows the effect of the 2′-amino modification on Mg²⁺ promoting the cleavage of preannealed ribozyme/substrate duplexes and on ribozyme/substrate global structure. Ribozyme (25 nM) and trace amount (1 nM) of 5′-³²P-labelled substrate were incubated for 5 min at 37° C. in 50 mM Tris HCl (pH 7.4). After incubation an aliquot from each sample was removed and analysed immediately by a 15% native gel electrophoresis (A), and then cleavage was initiated by adding Mg²⁺ to the remaining samples (B). Aliquots were removed at indicated time in seconds and analysed by electrophoresis on a 15% polyacrylamide gel with 7 M urea. C) Analysis of the global structure of the ribozyme/uncleavable substrate complexes in the presence of 5 mM Mg²⁺. The 2′-NH₂(2.1)-Rz or the Wt-Rz (25 nM) were annealed with the 5′-³²P-labelled uncleavable substrate (5 nM) at 37° C. for 10 min. in 50 mM Tris HCl, pH 7.4 and then analysed by gel electrophoresis in 10% native polyacrylamide gel with TB buffer containing 5 mM Mg²⁺ and visualised by a Phospholmager. The closed arrow indicates the unbound substrate, while the open arrow indicates the ribozyme/substrate duplexes.

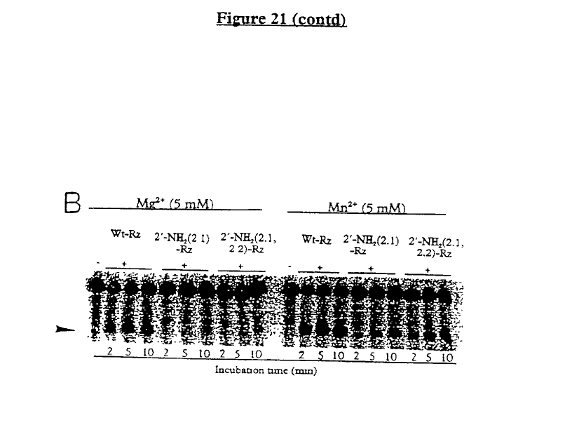

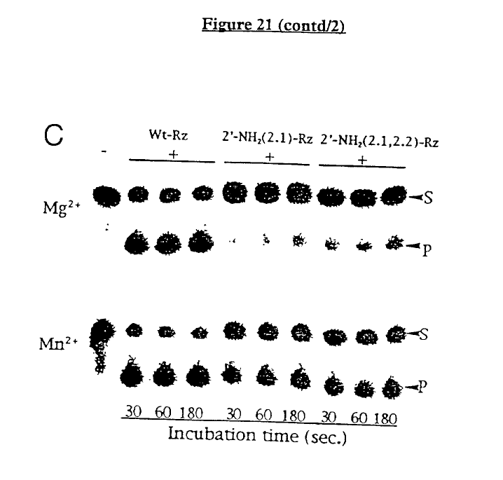

FIG. 21 shows cleavage activity of the ribozymes in the presence of various Mn²⁺ concentrations. A) Initial cleavage rates of the substrate with various Mn²⁺. Cleavage conditions are as in FIG. 18A. B) A representative example of the time course of the Wt-Rz and 2′-NH₂(2.1)-Rz mediated cleavage of the substrate in the presence of 5 mM Mg²⁺ or Mn²⁺ . C) Effect of Mg²⁺ or Mn²⁺ on the cleavage of the preannealed ribozyme/substrate complexes. Ribozymes (50 nM) and 5′-³²P-labelled substrate (5 nM) were incubated for 5 min at 37° C. in 50 mM Tris HCl (pH 7.4). Following incubation each sample was divided into two aliquots and cleavage was initiated by adding Mg²⁺ or to one of the samples. Aliquots were removed at the indicated time in seconds. Samples were analysed by electrophoresis on a 15% polyacrylamide gel with 7 M urea and visualised by a PhosphoImager.

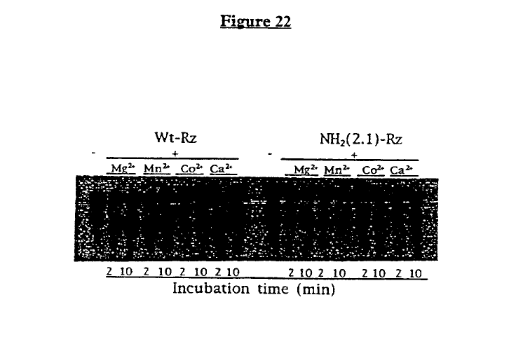

FIG. 22 shows cleavage activity of the Wt-Rz and 2′-NH₂(2.1)-Rz in the presence of various metal ions. Ribozymes (20 nM) and 5′-³²P labelled substrate (80 nM) were incubated for 10 min at 37° C. in 50 mM Tris HCl (pH 7.4) in the presence of 10 mM Mg²⁺, Mn²⁺, Co²⁺ or Ca²⁺. Aliquots were taken at indicated time, analysed by electrophoresis on a 15% polyacrylamide gel with 7 M urea and then visuallsed by a PhosphoImager.

### EXAMPLE 1

Preparation of 2′-Amino Derivatized Ribozymes and Testing for Catalytic Activity and Stability

Materials and Methods

In vitro RNA Synthesis of the Ribozymes

Both the unmodified and modified ribozyme having GUU corresponding to the codon number 4 in rat PKCα mRNA (Yoshitaka, 1988, Nucl. Acids Res., 16, p5911-5912) as the cleavage site were synthesized in vitro using DNA oligodeoxynucleotide and the T7 RNA polymerase as described previously (Sioud & Drlica, 1991, Proc. Natl. Acad. Sci., USA, 88, p7303-7307). In brief, to generate the ribozyme minigene, two overlapping half deoxynucleotides containing the T7 promoter sequence and the sequence coding for the catalytic centre and the flanking regions of the ribozyme were annealed and then extended with the Klenow fragment of DNA polymerase.

After extension, the DNA was polyacrylamide gel purified and then used as a template for in vitro transcription. The sequence of the PKCα Rz is 5′-GAAGGCCGGGUACUGAUGAGUCCGUGAGGACgAAACGUCAGCCAU-3′.

The non-cleaving mutant ribozyme (PKCα Rzm) was made by deleting the G12 from the catalytic core of the ribozyme as indicated by the lower case letter above.

The 2′-amino pyrimidine modified PKCα Rz was in vitro synthesized using the T7 RNA polymerase (Promega) or a mutant T7 RNA polymerase (Sousa & Padilla, 1995, EMBO J., 14, p4609-4621) kindly provided by Dr R. Sousa (University of Pittsburgh, Department of Biological Sciences, USA).

The mutant enzyme was partially purified from the overexpressing strain (Y639F) using a P-11 phosphocellulose column (Whatman).

The 2′-amino-2′-deoxyuridine and 2′-amino-2′-deoxycytidine (2′-amino pyrimidines) were purchased from Amersham and used as substrate for T7 RNA polymerase mainly as described in Aurup et al. (1992, Biochem., 31, p9636-9641).

The 5′-carboxyfluorescein-conjugated unmodified PKCα Rz used for the assessment of the transfection efficiency was chemically synthesized.

The mouse TNFα ribozyme used has the following sequence: 5′-GAAGAGGCUGACUGAUGAGUCCGUGAGGACGAAACAUAGGCACCG-3′.

RNA Substrates

PKCα—The short target RNA (5′-GAUGGCUGACGUUUACCCGGCC-3′) corresponding to the ribozyme site was synthesized by in vitro transcription using the following DNA template:

5′-TAATACGACTCACTATAGATGGCTGACGTTTACCCGGCC-3′
3′-ATTATGCTGAGTGATATCTACCGTCTGCAAATGGGCCGG-5′.

After transcription the target RNA was gel purified, dephosphorylated and then 5′-end labelled using [γ-³²P]ATP. The larger RNA substrate was internally labelled with [α-³²P]ATP during in vitro transcription.

The mouse TNFα short RNA substrate (5′-CGGUGCCUAUGUCUCAGCCUCUUC-3′) was chemically synthesized.

In vitro Cleavage Activity of the Unmodified and Modified Ribozymes

Cleavage reactions were performed at 37° C. in buffer containing 50 mM Tris-HCl (pH 7.4) and 20 mM MgCl₂, 10 nM ribozyme with 100 nM 5′-³²P end labelled target RNA. After the desired incubation time, cleavage products were separated by electrophoresis on 8 or 15% polyacrylamide gels with 7M urea and then visualized by autoradiography or PhosphorImager. Initial ribozyme cleavage rate and their k_{cat} were determined from Lineweaver-Burke plots (Trevor Palmer in “Understanding enzymes”, 3rd Edition, 1991, p1-399, Ellis Horwood press).

Ribozyme Stability in Serum

Internally labelled unmodified or pyrimidine modified PKCα Rz was incubated in RPMI supplemented with 10% FCS or human serum. Aliquots (10 μl) of the mixture were removed at various times, quenched with 20 ml TE/phenol/chloroform mixture, mixed and immediately frozen at −20° C. until use. At the end of the experiments, all samples were phenol extracted and analysed by 15% polyacrylamide gels with 7M urea.

Results

FIG. 1A shows TNFα complexed to its corresponding cleavage site within TNFα mRNA. TNFα ribozymes (unmodified and modified by 2′-amino modification of all pyrimidine bases) were prepared and both were found to cleave target RNA with comparable efficacy (FIG. 1B). Thus the use of a uniform 2′-amino modification did not affect ribozyme cleavage activity.

The effect of 2′-amino pyrimidine modification on the mouse TNFα ribozyme stability was investigated according to the methods (FIG. 1C). Half-lives were determined by the decrease in the radioactivity signal. In contrast to the unmodified PKCα Rz (t_{1/2}=0.3 min), the PKCα Rz with all 2′-amino pyrimidine nucleotides was remarkably stable with a half-life of >65 hours. The half-life time of the modified ribozyme in 10% freshly prepared human sera was approximately 52 hours.

Similar experiments were conducted on the protein kinase Cα ribozymes. FIG. 2A shows the PKCα complexed to its corresponding cleavage site within PKCα mRNA. The modified and unmodified PKCα Rz cleaved the short substrate with comparable efficacy (FIGS. 2B and C). In half-life studies (FIG. 2D) the unmodified PKCα Rz was found to have a half life of 0.25 min whereas the modified form had a half-life of >65 hours in FCS. In human sera the modified ribozyme had a half-life of approximately 55 hours.

### EXAMPLE 2

Effect of PKCα on Tumor Growth

Total PKC activity is enhanced in cancer cells (Couldwell et al., 1991, Neurosurgery, 29, p880-887; Deen et al., 1993, J. Neuro-Oncol., 16, p243-272). Furthermore, malignant gliomas are the third leading cause of death from cancer in individuals of 15 to 34 years of age as they fail to respond to irradiation, chemotherapy or immunotherapy (Nabel et al., 1993, Proc. Natl. Acad. Sci. USA, 90, p11307-11311).

The ability of PKCα ribozyme to block glioma cell proliferation in vitro and in vivo was investigated.

Materials and Methods

Cell Lines

The BT4Cn cell line is a subline of the BT4C cell line (Mella et al., 1993, J. Neuro-Oncology, 9, p93-104) and was obtained from fetal BDIX rat brain cells in culture, following transplacental ethylnitrosourea exposure in vivo. These cells were provided by Dr Rolf Bjerkvig (Institute of Anatomy and Cell Biology, the University of Bergen, Norway). Cells were grown in RPMI supplemented with 10% fetal calf serum (FCS).

Western and Northern Blot Analysis

Extraction of protein from cultured cells was performed as previously described (Sioud, 1994, J. Mol. Biol., 242, p619-629; Sioud & Jespersen, 1996, J. Mol. Biol., 257, p775-789). Equal amount of proteins (15 μg/lane) were analysed by SDS gel electrophoresis with a 10% polyacrylamide separating gel. Following electrophoresis, proteins were transferred to nitrocellulose membrane and immunoblotted with rabbit IgG polyclonal anti-PKCα, anti-PKCδ, anti-PKCγ, anti-Bcl-xL, anti-Bcl-2 or anti-Bax antibodies (Santa Cruz Biotechnology), and then visualised by the ECL-system (Amersham) using horseradish peroxidase conjugated anti-rabbit IgG (Sigma). Total RNA was prepared according to Chomczynski & Sacchi (Anal. Biochem., 1987, 162, p156-159) and analysed by 1% agarose gel (20 μg/lane). Subsequently it was blotted onto a nylon membrane (Amersham) and hybridised to a 5′-³²P-labelled antisense oligonucleotide specific for PKCα (5′-GGAGTCGTTGGCCGGGTAAACGTCAGCCAT-3′).

Transfection Experiments

The cells were transfected with cationic liposomes (DOTAP, Boehringer Mannheim, Germany) at a concentration of 30 μg/ml either alone or complexed with the test molecules as described previously (Sioud, 1994, J. Mol. Biol., 242, p619-629). In order to study the kinetics of uptake into BT4Cn, a carboxyfluorescein-conjugated RNA/DNA chimeric PKCα ribozyme directed to the same site was delivered to the cells and the uptake was investigated with flow cytometer and epifluorescence microscope.

MTT (Tetrazolium) Assay

BT4Cn cells were re-suspended in RPMI containing 10% FCS at a concentration of 2×10⁴ cells/ml and 100 μl aliquots (2×10³ cells) were plated into 96-well, flat-bottom tissue culture plates. The plates were incubated at 37° C. for at least 6 hours prior to transfection. After 48 hours transfection time, stock MTT solution (10 μl per 100 μl medium) was added to all wells, plates were incubated at 37° C. for 4 hours, a 100 μl of isopropanol/HCl was added to all wells and then the plates were read using a test wavelength of 570 nm and a reference wavelength of 630 nm.

Animals and In vivo Experiments

Inbred BDIX rats of both sexes (300±50 g) were housed two or three to a cage under conditions meeting AALAC-International standards. Rats were tested negative for parasitical, bacterial and viral agents according to the recommendations of the Federation of European Laboratory Animal Science Associations (FELASA). Animal experimentation was performed under conditions meeting AALAC-International standards. The BT4Cn cells (2×10⁵) were inoculated s.c. into the flank of the rats. A single dose (50 μl) of the liposomes containing the 2′-amino pyrimidine modified PKCα Rz (200 μg), the PKCα Rzm (200 μg), or only DOTAP (10 μg) was injected into the centre of the tumour when the tumour size reached 4 to 5 mm in diameter, approximately 3 weeks after cell inoculation. All injected solutions were adjusted to 0.9% NaCl prior to injection. The injection of the test molecules into the tumour was done under anaesthesia with s.c. injection of fentanyl (0.15 mg/kg) and midazolam (2.5 mg/kg). Tumour size was assessed at day 8, 15 and 20 following ribozyme injection. When the tumours in rats injected only DOTAP reached a maximum size, all animals were killed by i.p. injection of pentobarbitone at 100 mg/kg, tumours were removed, weighed, frozen in liquid N₂ and kept at −70° C. until use.

Immunohistochemistry

To evaluate the expression of PKCα and Bcl-xL, ethanol fixed sections from the periphery of the tumour were blocked with bovine serum albumin (1% BSA in PBS) for 30 min and then immunoreacted with a 1/20 dilution of a rabbit IgG polyclonal antibody directed against the PKCα or the Bcl-xL (Santa Cruz Biotechnology) in PBS containing 1% BSA. After 3 washed in PBS, positive staining was detected by a 1/20 dilution of FITC-conjugated goat anti-rabbit IgG (DAKO). Representative sections were also stained with hematoxylin/eosin.

Results

The delivery by transfection of PKCα Rz into glioma cells in the form of a 5′-carboxyfluorescein-conjugated DNA/RNA chimeric PKCα ribozyme using DOTAP was assessed by flow cytometry and epifluorescence microscope analysis (FIGS. 3A, B and C). Efficient delivery was observed.

The cationic lipid DOTAP was then used as a vehicle for delivery of the test molecules (5 μl) to establish the effect of the modified ribozyme on the proliferation rate of the aggressive cell line BT4Cn. Proliferation was reduced by 77% and 45% in the presence of the modified ribozyme or in the presence of its mutant form (PKCα Rzm), respectively (FIG. 4A). Assuming normal distribution and independence of each treatment, a significant difference between control, PKCα Rz- and PKCα Rzm-treated cells was found (P<0.0001) by one-way analysis of variance. Notably, the antiproliferative effect of the active ribozyme was significantly higher than its mutant form (P<0.0004) as assayed by Fisher's protected least square difference (LSD) test. At the concentration used, no apparent cytotoxicity of DOTAP was detected on glioma cell survival as assessed by Trypan Blue exclusion test.

To test the ability of PKCα Rz to specifically reduce PKCα gene expression and the effect of such down regulation on the expression of other cell survival molecules, the PKCα, the Bcl-XL and Bax protein amounts in both DOTAP- and test molecule-treated cells were determined by immunoblotting. Densitometric scanning indicated that in modified PKCα Rz-treated cells, the PKCα and the Bcl-XL signals were reduced by 70% and 75%, respectively (FIG. 4B). The detection of the PKCα protein in the ribozyme-transfected cells may not be surprising, since it has a longer half-like in glioma cells (25 hours). The observed inhibition effect was specific, as synthesis of the death agonist Bax protein was not affected by the ribozyme treatment. The mutant ribozyme, PKCα Rzm, reduced the PKCa and Bcl-XL signals by 50% and 35%, respectively.

To determine if PKCα Rz could eliminate its mRNA target following transfection, total RNA from DOTAP-treated cells (control), PKCα Rz-treated cells and PKCα-Rzm-treated cells were extracted and analysed by Northern blotting using a PKCα probe. The PKCα Rz reduced its cellular target mRNA by 97% (FIG. 4C), whereas the mutant ribozyme reduced the PKCα mRNA level to a lesser degree (40%). Furthermore, down regulation of the PKCα by the ribozyme was found to induce apoptosis (cell death) in glioma cells (FIG. 4D).

To determine the functional significance of the PKCα isoform during tumour growth, inbred syngeneic BDIX rats were inoculated subcutaneously with BT4Cn cells to produce tumours which were injected with the modified or mutant ribozymes as described in the methods section. Rats were killed at day 20. The kinetics of tumour growth following PKCα Rz inoculation are presented in FIG. 5A and B. The modified PKCα Rz inhibited tumour growth as assessed either by tumour size (A) or by tumour weight at day 20 (B, as a representative example). The average tumour weights (in grams)±s.d. at 20 days post-treatment for DOTAP, PKCα Rz and PKCα Rzm were 14±3 (n=5), 0.25±0.15 (n=8) and 3.5±1.3(n=8), respectively. Notably, the growth rate of mutant ribozyme-treated tumour was reduced, whereas it was nearly blocked in ribozyme-treated tumour when compared to the DOTAP-treated tumour (control).

At day 20, tumours were removed and immediately frozen in liquid N₂. Hematoxylin/eosin staining of frozen sections (FIGS. 5C and D) revealed that control tumours consisted of a homogeneous mass of viable cells as illustrated in FIG. 5D. In contrast, few cells and large areas of necrosis were seen in PKCα Rz-treated tumours (FIGS. 5E and F). In two rats inoculated with the PKCα Rz, the BT4Cn tumour started to grow at day 15 following the first inoculation. Interestingly, a second inoculation blocked tumour growth. These two rats were not included in the data presented in FIG. 5A. A combination of the present PKCα Rz with a second ribozyme directed to the first GUC within the PKCα translated region completely eliminated the tumour in two treated rats.

To determine whether the ribozyme-treated tumours would exhibit reduced expression or a lack of expression of the PKCα and the Bcl-XL, tumour sections from the periphery of the tumour were analysed by immunohistochemistry using PKCα or Bcl-XL specific antibodies. Notably, the ribozyme treated tumour contained some viable cells, as their cytoplasmic and nuclear membranes are intact. The expression of the PKCα and the Bcl-XL was reduced in these cells as compared to DOTAP-treated tumour cells (FIG. 6).

Similar experiments were conduced with a human PKCα 2′-amino modified ribozyme directed to the same site (FIG. 7A). This was found to induce apoptosis in human glioma cell lines (FIG. 7B).

### EXAMPLE 3

Effect of 2′-Amino Modification on VEGF Ribozyme and Effect on Tumor Growth

Experiments were performed essentially as described in Examples 1 and 2. A ribozyme directed to the vascular endothelial growth factor was designed (FIG. 8A). The 2′-amino modified ribozyme cleaved its target RNA with similar efficacy as its unmodified version (FIG. 8B). Furthermore it was found to inhibit tumour growth in vivo (FIG. 8C).

Similar to the above rat VEGF ribozyme, a human ribozyme directed to the same site was also found to tolerate the complete 2′-amino modification.

### EXAMPLE 4

High Cleavage Activity and Stability of Hammerhead Ribozymes with a Uniform 2′-Amino Pyrimidine Modification

Materials and Methods

In vitro RNA Synthesis

Unmodified and 2′-amino pyrimidine modified ribozymes were synthesised by in vitro transcription using DNA oligodeoxy-nucleotides and the T7 RNA polymerase as previously described (Sioud et al. (1991), Proc. Natl. Acad. Sci. 88: 7303-7307; Sioud, M. (1994), J. Mol. Biol. 242: 619-629). Following transcription, intact ribozymes were gel-purified, eluted, ethanol-precipitated, washed with 70% ethanol, dried and resuspended in water and their concentration was determined by measurement of absorbency at 260 nm. The 2′-amino modified nucleotides were obtained from Amersham (Little Chalfont, United Kingdom) and used as substrates for the T7 RNA polymerase as described by Aurup et al. (1992), Biochemistry 31: 9636-9641. Target RNAs corresponding to the vascular endothelial growth factor (VEGF) ribozyme cleavage site was synthesised by in vitro transcription of a synthetic DNA template with the T7 RNA polymerase and subsequently gel-purified. After transcription, the gel-purified substrate RNA was dephosphorylated by alkaline phosphate and then 5′-end labelled using T4 polynucleotide kinase and [γ-³²P] ATP. The RNA substrates for the mouse tumour necrosis factor α (TNFα) was chemically synthesised. The unmodified, modified TNFα ribozyme with 2′-amino uridine at position 2.1 and 2.2 and the RNA substrate were chemically synthesised by Dr. Phil Hendry and Maxine McCall (CSIRO, Sydney, Australia).

In vitro Cleavage Activity of the Unmodified and Modified Ribozymes

Cleavage reactions were performed at 37° C. in buffer containing 50 mM Tris-HCl (pH 7.4) and 10 mM MgCl₂. Cleavage products were separated by electrophoresis on a 15% polyacrylamide gel containing 7M urea and scanned on a Molecular Dynamics PhosphoImager. Initial ribozyme cleavage rates and k_{cat} were determined from Lineweaver-Burk plots.

Ribozyme Stability Analysis in Fetal Calf Serum (FCS)

[α-³²P] ATP internally labelled ribozymes were incubated in medium containing 10% FCS. Aliquots of the mixture were removed at various times, quenched with phenol/chloroform mixture and frozen until use. All samples were phenol extracted and analysed on 15% polyacrylamide gel with 7 M urea and scanned by a Molecular Dynamics PhosphoImager.

Results

In Trans Cleavage Activity of Ribozymes with a Complete 2′-amino Pyrimidine Modification

Recent experiments have shown loss in cleavage activity of totally 2′-amino pyrimidine substituted ribozymes. In particular, where all pyrimidines had been replaced by their 2′-amino analogs only 1.9% cleavage activity was retained (Pieken et al. (1991), Science 253: 314-317). To our surprise, when the cleavage activity of a 2′-amino pyrimidine modified rat PKCα ribozyme was investigated, it was found to be 40 to 60% of the unmodified ribozyme activity. Indeed, the unmodified and the modified PKCα ribozyme cleaved the short RNA substrate with an apparent turnover number of 0.32 min⁻¹ and 0.20 min⁻¹, respectively. Most importantly, the modified ribozyme blocked tumour growth in vivo.

To see whether this similarity in cleavage also applied to other ribozymes, all pyrimidine nucleotides in a TNFα ribozyme were substituted with their 2′-amino analogues (see FIG. 1A and Example 1). Both ribozymes cleaved the RNA substrate with comparable efficacy (FIG. 1B). The high cleavage activity of the modified TNFα ribozyme is in disagreement with the data reported by Pieken et al. where a chemically and uniformly modified 2′-amino pyrimidine ribozyme showed almost no cleavage activity (Pieken, supra). As hammerhead ribozymes have virtually the same catalytic core, the inhibition of cleavage in their case must be due to the presence of 2′-amino pyrimidines in helix I and/or III.

The presence of 2′-amino groups at positions 2.1 and 2.2 inhibits the ribozyme cleavage activity To evaluate the effect of the 2′-amino groups on the ribozyme cleavage activity as a first step, we investigated the effect of a 2′-amino group at position 2.1 (Hertel et al. (1992), Nucleic Acids Res. 21: 2809-2814) A TNFα ribozyme identical to the one shown in FIG. 1, but with a cytidine (C) at position 2.1 and a guanidine (G) in the corresponding position in the substrate was designed. Complete 2′-amino pyrimidine modification reduced the ribozyme cleavage activity by approximately 6-fold. This data would suggest a negative effect of the 2′-amino group at position 2.1 on the ribozyme catalyst potency. Complete 2′-amino pyrimdine substitution in a second TNFα ribozyme containing pyrimidines at positions 2.1 and 2.2 reduced the ribozyme cleavage activity by almost 7 to 8-fold.

To gain further insight into the effect of the 2′-amino groups at positions 2.1 and 2.2 on ribozyme cleavage activity we have performed a selective modification. A TNFα ribozyme identical to the one shown in FIG. 1, but with 2-amino uridines at only positions 2.1 and 2.2 was chemically synthesised (FIG. 13A). See also FIG. 15 for standard numbering of a similar hammerhead ribozyme. Interestingly, such specific modification reduced the ribozyme cleavage activity by almost 8-fold (FIG. 13B and C, as a representative example).

Design of Ribozymes that can be Totally 2′-amino Pyrimidine Substituted

Although the catalytic potency of hammerhead ribozymes might be in part influenced by their secondary structures following 2′-pyrimidine modifications, analysis of several ribozymes indicated that ribozymes containing purines at position 2.1, 2.2 and 15.2 have their catalytic activities either unaffected or slight affected by the 2′-amino pyrimidine modifications. To illustrate this observation, ribozyme directed against VEGF was designed and its in vitro cleavage activity was investigated. In addition to many biological roles, VEGF plays a crucial factor in tumour angiogenesis and metastasis (Zetter, B. R. (1998), Annu. Rev. Med. 49: 407-424). FIG. 8A (Example 3) shows the VEGF ribozyme complexed with its corresponding cleavage site within the rat VEGF mRNA sequence. As can be seen, the ribozyme was designed to contain no pyrimidines in helix I, while position 15.2 contains a purine (G). The 2′-amino pyrimidine modified ribozyme cleaved the target RNA with almost the same efficacy as the unmodified ribozyme (FIG. 8, Example 3). The apparent turnover k_{cat} for the unmodified and modified ribozymes were found to be 1.4 (±0.15) min⁻¹ and 1.32 (±0.12) min⁻¹, respectively.

In vitro Stability of the Unmodified and Modified Ribozymes

One of the major problems associated with exogenous delivery of ribozymes is their sensitivity to nucleases present in biological fluids. In this respect, pyrimidines in hammerhead ribozymes have been shown to be a major site for nucleases. Thus, we have investigated the effect of the 2′-amino pyrimidine modification on the VEGF ribozyme stability. Internally labelled unmodified or modified ribozyme were incubated in cell culture medium containing 10% FCS. In contrast to the unmodified ribozyme (t_{½}=0.1 min) the ribozyme with all 2′-amino pyrimidine nucleotides was found to be stable in 10% FCS. No significant degradation was observed following 48 hours incubation time (FIG. 14). Similar stability results were obtained with the other ribozymes.

### EXAMPLE 5

Ribozyme Inhibition of the Protein Kinase Cα Triggers Apoptosis in Glioma Cells

This Example demonstrates that a ribozyme specific for the human protein kinase Cα (PKCα), a classical PKC isoform, induces cell death in glioma cell lines. This cell death was identified as apoptosis by morphologic alterations and endonucleosomal DNA fragmentation. The inhibition of PKCα gene expression by the ribozyme resulted in a significant reduction in Bcl-x_{L} gene expression, a protein that inhibits apoptosis and is overexpressed in glioma cells. Taken together, the data suggest that the PKCα ribozymes are a potent inducer of apoptosis in glioma cells, which may act through suppressing Bcl-x_{L} gene expression and/or activity.

This Example investigates the molecular mechanisms by which PKC isoform-specific inhibitors inhibit glioma cell proliferation. The results demonstate that inhibition of PKCα leads to a decrease in Bcl-X_{L} gene expression and consequent induction of apoptosis.

Materials and Methods

Cell Lines

Human T98G and U87MG glioblastoma cell lines were obtained from American Tissue Type Culture (ATCC), and grown in DMEM medium supplemented with 10% fetal bovine serum (FBS) according to the instructions of ATCC.

Western Analysis

Cytoplasmic extracts were prepared from control and test molecule-treated cells according to Sioud M. (1994), Interaction between tumour necrosis factor a ribozyme and cellular proteins, J. Mol. Biol. 242: 619-629; Sioud et al. (1996), Enhancement of hammerhead ribozyme catalysis by glyceraldehyde-3-phosphate dehydrogenase, J. Mol. Biol. 257: 775-789. Extracts (15 μg/lane) were separated by electrophoresis on a 10% polyacrylamide gel under denaturing conditions. Proteins were transferred to nitrocellulose membrane and immunoblotted with a rabbit IgG polyclonal anti-PKCα, anti-Bcl-x_{L} or anti-Bax antibodies (Santa Cruz Biotechnology), and visualised by the ECL-system (Amersham) using horseradish peroxidase conjugated anti-rabbit IgG (Sigma).

Subcellular Fractionation

Cells were washed in ice-cold phosphate buffered saline (PBS) and resuspended in buffer A (5 mM Tris-HCl, pH 8, 0.5 mM EDTA, 75 mM sucrose and proteinase inhibitors) and then sonicated 4 times, 15 seconds each. Complete cell lysis was confirmed by microscopy. Nuclei were pelleted by centrifugation at 2000 rpm for 5 min at 4° C. in a microcentrifuge. The supernatants were centrifuged at 40,000 rpm for 30 min. at 4° C. in a Beckman ultracentrifuge. Each supernatant was collected and used as the cytosol fraction. The membrane pellets were washed 3 times with PBS, solubilised in buffer A containing 1% Triton×100 for 15 min at 4° C. and then centrifuged at 15,000 rpm for 10 min at 4° C. in a microcentrifuge. Supernatants were used as the membrane fraction. In all cases, protein concentrations were determined using the protein assay kit (BioRad).

MTT (Tetrazolium) Assay

Cells were resuspended in DMEM containing 10% FBS at a concentration of 2×10⁴ cells/ml and 100 μl aliquots (2×10³ cells) were plated into 96-well, flat-bottom tissue culture plates. The plates were incubated at 37° C. for at least 6 hours to allow recovery of the cells from trypsination. Following incubation, cells were transfected with the test molecules in complete medium using DOTAP as described previously (Sioud, 1994). After 48 hours transfection time, stock MTT solution (10 μl per 100 μl medium) was add to all wells, and the plates were incubated at 37° C. for 4 hours Acid-isopropanol (100 μl of 0.04 N HCl in isopropanol) was added to all wells and mixed thoroughly to dissolve the formed crystals, the plates were read using a test wavelength of 570 nm and a reference wavelength of 630 nm.

In vitro RNA Synthesis

An asymmetric 2′-amino modified ribozyme having a cleavage site the GUU corresponding to the codon number 4 within the human PKCα mRNA were synthesised by in vitro transcription using DNA oligodeoxynucleotide and the T7 RNA polymerase as described previously. (Sioud M, Drlica K, (1991), Prevention of HIV-1 integrase expression in E. coli by a ribozyme. Proc. Natl. Acad. Sci. USA 88: 7303-7307). In brief, to generate the ribozyme minigene, two overlapping half deoxynucleotides containing the T7 promoter sequence and the sequence coding for the catalytic center and the flanking regions of each ribozyme were annealed and then extended with the Klenow enzyme. After extension, the DNA was polyacrylamide gel purified and then used as template for in vitro transcription. Following transcription RNA as gel purified. The ribozyme sequence is: 5′GGGAACUGAUGAGUCCGUGAGGACgAAACGUCAGCCAUGG3′. Ribozyme with only antisense activity, mutant ribozyme, was made by deleting the G12 from the catalytic core as indicated by lower case letter.

Total RNA Preparation and RT-PCR

Total RNA was prepared from control and test molecule-treated cells according to Chomczynski et al., (1987), Single step method of RNA isolation by acid guanidinium thiocyanate phenol chloroform extraction., Anal. Biochem. 162: 156-159, and one 1 μg was reverse transcribed using the first strand cDNA synthesis kit and oligo dT primers as recommended by the manufacturer (Pharmacia, Uppsala, Sweden). Polymerase chain reaction was performed on entire cDNA product by using Taq DNA in a gene Amp PCR system 2400 (Perkin-Elmer/Cetus) using primers specific for the PKCα insoform. Following 30 cycles of amplification, PCR products were separated in a 1.5% agarose gel and visualized by staining with ethidium bromide. As a control, actin was co-amplified using specific primers.

TUNEL-reaction

A commercially available in situ cell death fluorescein detection kit based upon terminal deoxynucleotidyl transferase (TdT)-medicated dUTP-FITC nick end labelling (TUNEL) was used (Boehringer). Briefly, cells were washed with PBS and fixed in 4% paraformaldehyde solution in PBS. Then after washing with PBS cells were permeabilised with 0.1% Triton×100 in 0.1% sodium citrate for 2 min at 4° C. Following washing with PBS, cells were incubated with the TUNEL reaction for 30 min, washed with PBS and then analysed by flow cytometry.

Detection of DNA Fragmentation Following Ribozyme Treatment

After ribozyme treatment, cell pellets were lysed in 0.02% N-lauryl-sarcosine (Sigma) in 50 μl TE buffer. Ribonuclease A was added and lysates were incubated at 37° C. for 30 min. Thereafter, proteinase K was added and samples were incubated at 37° C. for another 60 min. Following incubation, the resultant crude DNA gene expression was not affected by this treatment (FIG. 9C). The level of PKCA decreased in this PTA-treatment cells. Long term PTA (100 nM) stimulation of glioma cells induced PKCα down regulation, but not depletion (data not shown), suggesting an active de novo synthesis.

Effect of the PKCα Ribozyme on Cell Proliferation and on PKCα Gene Expression

To evaluate the effect of the PKCα isoform on the human glioma cell proliferation, we have targeted its expression by the human PKCα ribozyme. As shown in FIG. 10A, the proliferation rate of the glioma cells was reduced by 90% (±5%) and 55% (±10%) in the presence of the ribozyme or its mutant form, respectively. A significant difference between DOTAP-, ribozyme- and mutant ribozyme-treated cells was found (P<0.0007). The antiproliferative effect of the active ribozyme was significantly higher than its mutant form (P<0.0003). Notably, the effect of the mutant ribozyme on cell proliferation is also significant (P<0.0026). The inhibition effect of the mutant ribozyme is more likely to be due to its antisense activity.

To see whether the inhibition of cell proliferation after ribozyme treatment was reflected at the protein level, cell extracts obtained from control and test molecule-treated cells were subjected to Western blot analysis. In ribozyme-treated cells the amount of PKCα was reduced by approximately 73% (±5%) (P<0.0001) and that of Bcl-c_{L} was reduced by 90% (±15%) (P<0.0001) as compared to controls. This result would suggest a possible interaction between PKCα and Bcl-x_{L} proteins. A significant inhibition (50% ±10% of PKCα gene expression was also seen in mutant ribozyme-treated cells (P<0.002). Ribozyme inhibition was isotype specific since the PKCα levels were unaffected by any of the treatments (FIG. 10B). The detection of the PKCα protein in the ribozyme-treated cells may not be surprising, since it has a long half-life in glioma cells (>25 hours). Analysis of the PKCα mRNA in DOTAP-, mutant ribozyme-, and ribozyme-treated cells by RT-PCR (FIG. 10C) shows a dramatic reduction in PKCα signal in ribozyme-treated cells as compared to mutant ribozyme (PKC Rzm). This result would indicate that the inhibition effect of the ribozyme on PKCα gene expression is due to its cleavage activity of the mRNA.

Effect of Ribozyme-medicated Loss of PKCα Protein on Induction of Apoptosis

Morphological examination of glioma cell lines treated with the PKCα ribozymes indicated alteration in cellular morphology. Cells became rounded and displayed condensation of the nuclear chromatin as shown in FIG. 11B. These morphological changes are reminiscent of apoptosis. The extent of apoptosis in the presence or absence of the ribozyme was assessed by the percentage of apoptotic nuclei visualised by propidium iodide staining (FIG. 11C). In cells treated with ribozyme for 48 hours, the percentage of apoptotic nuclei was 87% as compared to only 5% in control cells. A significant fraction (30%) of mutant ribozyme-treated cells are apoptotic. That ribozyme-treated glioma cells were killed by apoptosis was confirmed by the use of the deoxynucleotidyl transferase-mediated dUTP nick end labelling (TUNEL) assay. This method is based on the fact that apoptotic cells contain free 3′-end of double-stranded DNA due to the endonuclease digestion of genomic DNA at the nucleosomal intervals. FITC-conjugated dUTP molecules were added to these 3′ end using the terminal deoxynucleotidyl transferase enzyme (FIG. 12D). As can be seen, all ribozyme-treated cells were in apoptotic stage. The induction of cell death in human glioma cells following ribozyme treatment was further confirmed by the presence of a DNA ladder which directly reflects the endonucleotic cleavage of chromosomal DNA typically associated with the apoptotic process (FIG. 12, lane 3).

Conclusions

This study demonstrated that inhibition of endogenous PKCα synthesis by a ribozyme induces apoptosis in cultured malignant gliomas, supporting an essential survival function for PKCα in these cells. The inhibition effect is specific, since the expression of the PKCδ isoform was not affected by the ribozyme treatment. Furthermore, the study indicates that the expression and/or the activity of the cell survival molecules Bcl-x_{L} is under the control of PKCα signal pathway. This observation is important, because it links the PKCα isoform with apoptosis.

Free ribozymes or capsules containing ribozymes can be injected into the tumour. This strategy may offer many advantages over systemic therapy, since it would ensure a high concentration of the drug within the tumour and more importantly would be less toxic to normal cells.

In conclusion, we have demonstrated that a selective inhibition of PKCα gene expression by a ribozyme decreases proliferation of human glioma cell lines in vitro by activating the apoptotic process. Thus, our data demonstrate for the first time that the machinery of apoptosis in cancer cells can be targeted specifically by ribozymes and suggest a potential interaction between PKCα and the Bcl-x_{L} protein. preparations were analysed by electrophoresis on a 1% agarose gel and visualised by ethidium bromide staining.

Statistical Analysis

Each experiment was performed at least 4 times. Statistical significance of ribozyme and mutant ribozyme effects on cell proliferation, PKC and Bcl-x_{L} gene expression were assessed by unpaired Student's t-test.

Results

Human Glioma Cell Lines Upregulate the Expression of PKCα and Bcl-x_{L} Proteins

The experiment was designed to determine whether the upregulation of the PKCα and the BCl-x_{L} gene expression is also a property of human glioma cells. As illustrated in FIG. 9A, the T98G and U87MG cell lines overexpress the PKCα and the Bcl-x_{L} proteins. The expression of Bcl-2 protein by both cell lines was very weak and in many cases undetectable (data not shown). A significant fraction (15% of the PKCα was found to be associated with membrane fraction (FIG. 9B). As expected, the Bcl-x_{L} is merely a membrane bound protein.

Because many gene products have been shown to be under the control of the PKC signal pathway therefore we investigated whether the activation of PKC by phorbol esters (e.g. PTA) would increase Bcl-x_{L} gene expression in glioma cells. In principle, binding of TPA to the amino-terminal regulatory regions of some PKC isoforms, in particular PKCα, would induce conformational changes, resulting in their activation, membrane translocation and sensitivity to proteoletic cleavage. The time course for PKC activation, depletion and their de novo synthesis following TPA stimulation varies significantly with cell types. Exposure of U87MG cells to TPA for 5 hours led to a 3-fold increase in Bcl-x_{L} gene expression, while the Bax

### EXAMPLE 6

Substitution of the 2′- Hydroxy Group at Position 2.1 by an Amino Group Interferes with an Important Mg²⁺ Binding Site Required for Efficient Cleavage by Hammerhead Ribozyme

It has been demonstrated in the previous Examples that hammerhead ribozymes can be fully substituted with 2′amino pyrimidines without detriment to the catalytic activity, provided that positions 2.2 and/or 2.1 are not modified (see FIG. 15 for numbering of the ribozyme). Therefore, the potential molecular mechanisms by which 2′-amino groups at these positions inhibit ribozyme cleavage rate were investigated using site specific modification. In the presence of Mg²⁺, the 2′-amino modification at positions 2.2 and/or 2.1 had no significant effect on substrate binding. However, it dramatically inhibited the ribozyme cleavage activity. Analysis of the ribozyme cleavage rates in the presence of various Mg²⁺ concentration revealed that Mg²⁺ binding is inhibited by the 2′-amino group at position 2.1. Additionally, the 2′-amino modified ribozyme/substrate complexes exhibited a significant difference in electrophoretic mobility when compared to the complexes formed by its unmodified version, suggesting that the Mg²⁺ promoted folding is inhibited by the 2′-amino group. Surprisingly, the cleavage rate of the modified ribozyme was substantially increased when Mg²⁺ ions were replaced by the thiophilic Mn²⁺ ions, whereas only a moderate enhancement occurred with its unmodified version. In contrast, replacement of Mg²⁺ by the thiophilic Co²⁺ ions did not restore the ribozyme cleavage activity, indicating that the rescue effect of Mn²⁺ ions is specific.

The trans acting hammerhead ribozyme described by Haseloff and Gerlach (Nature, (1988), 334: 585-591) consists of three helical stems including nine conserved nucleotides that are responsible for the formation of a catalytically active domain. Cleavage of the substrate occurs via internal transesterification involving the 2′-hydroxyl adjacent to the suicide bond. This results in the formation of a 2′-3′ cyclic phosphate on the 5′ fragment and a free 5′-hydroxyl on the 3′ fragments. Although no protein is required, a divalent cation cofactor such as Mg²⁺ and Mn²⁺ are necessary for the cleavage reaction.

Materials and Methods

Ribozymes and RNA Substrates

The RNA substrates, unmodified and modified TNFα ribozyme with 2′-amino uridine at position 2.2 and 2.2 were chemically synthesised by Dr. Phil Hendry and Maxine McCall (CSIRO, Sydney, Australia). The TNFα ribozyme with 2′-amino group at position 2.1 and the uncleavable RNA substrate were synthesised by integrated DNA Technology inc. (USA). All ribozymes and RNA substrates were polyacrylamide gel purified. RNAs were 5′-end labelled using T4 polynucleotide kinase and [γ-³²P] ATP. The partial alkaline treatment of ribozymes was made by incubation of 5 nM of 5′-³²P-labeled ribozymes in 50 mM sodium bicarbonate (pH 9.2) at 95° C. for 10 min.

In vitro Cleavage Activity of the Unmodified and Modified Ribozymes

Cleavage reactions were performed at 37° C. in buffer containing 50 mM Tris HCl (pH 7.4). The various concentrations of the metal ions and cleavage conditions are indicated in the figure legends. Cleavage products were separated by electrophoresis on a 15% polyacrylamide gel containing 7 M urea and quantitated by using a Molecular Dynamics PhosphoImager. Initial ribozyme cleavage rates were determined from the initial stage slopes of the curves for the time-course relation of the reaction. The k_{cat}'s were calculated from Eadie-Hoftee plots.

Cleavage of the Preannealed Ribozyme/substrate Duplexes

5′-³²P-labelled substrate was annealed to 10 fold excess of ribozyme in 50 mM Tris HCl (pH 7.4) at 37° C. for 5 minutes. Following annealing, an aliquot was removed from each sample and analysed immediately by 15% native gel electrophoresis. Mg²⁺ or Mn²⁺ ions were added to the rest of the preannealed ribozyme/substrate to start the cleavage reaction. Samples were removed at 30, 60 and 120 seconds and quenched immediately in stop solution containing formamide and EDTA. Samples were analysed by electrophoresis on a 15% polyacrylamide gel containing 7 M urea, and quantitated by using a Molecular Dynamics PhosphoImager.

Binding Experiments

Various concentrations of the 5′-labelled substrate were incubated with the same concentration of ribozymes. Following incubation samples were analysed by 15% native gel electrophoresis. To determine the K_{d} a trace amount of 5′-³²P-labelled substrate (1 nM) was incubated with various concentrations of each ribozyme. Free and bound substrate were quantitated by using a Molecular Dynamics PhosphoImager.

Analysis of the Global Structure of the Ribozymes Complexed with an Uncleavable RNA Substrate

5′-³²P-labelled uncleavable RNA substrate (5 nM), in which the ribose of the C17 was changed to a deoxyribose, was annealed to 5 fold excess of ribozyme in 50 mM Tris HCl (pH 7.4) at 37° C. for 10 minutes in 10 μl volume. After annealing, 0.5 μl of 50% glycerol solution was added and samples were immediately analysed by 10% native polyacrylamide gels at room temperature for 6 hours at 100 V in TB buffer containing 5 mM Mg²⁺. Gels were analysed by a Molecular Dynamics PhosphoImager.

Results and Discussion

2′-amino Group at Position 2.1 Inhibits Ribozyme Cleavage Activity

Analysis of the cleavage activity of many ribozymes with uniform 2′-amino pyrimidine modification indicates that ribozymes containing purines in helix I, especially at position 2.1, can be totally substituted by 2′-amino pyrimidines without significant loss of their cleavage activity. Indeed, replacement of all pyrimidines in PKCA TNFα or VEGF ribozyme yielded nuclease resistant ribozymes with sustained cleavage activity. From these observations it appears that the presence of 2′-amino group near the cleavage site has a significant inhibition effect upon ribozyme cleavage activity. In this respect, we have noted a dramatic decrease in a TNFα ribozyme cleavage activity for the A^{2.1} and G^{2.2}→2′-amino U^{2.1} and 2′-amino U^{2.2}, respectively. The observed inhibition effect could originate from the presence of a 2′-amino group at position 2.1 only, since it is locked near the cleavage site. To investigate which position is involved, and to provide molecular information about the mechanism of inhibition, a TNFα ribozyme with 2′-amino uridine at position 2.1 was also designed. FIG. 16A (and FIG. 15 for standard numbering) shows the base-pairing of the ribozymes with their corresponding target RNAs.

To see that a correct modification had been introduced, 5′-labelled ribozymes were analysed by partial alkaline 30 hydrolysis. The partial degradation of the unmoved ribozyme is shown in lane 2. Degradation of the TNFα ribozyme with 2′-amino groups at position 2.1 revealed that this position is protected from cleavage (lane 4). Positions 2.1 and 2.2 are protected in the TNFα ribozyme with 2′-amino group at position 2.1 and 2.2 (lane 6).

Having confirmed the presence of the modified nucleotides within the ribozyme, in the next experiment we have investigated their cleavage activity (FIG. 17A and B). As shown, the 2′-amino modification has hampered the ribozyme cleavage activity. When cleavage reactions were performed in the presence of 10 mM Mg²⁺, the apparent turnover number (k_{cat}) for unmodified ribozyme (Wt-Rz), the ribozyme with a 2′-amino U^{2.1} [2′-NH₂ (2.1)-Rz] and the ribozyme with a 2′-amino U²¹ and a U²² [2′-NH₂(2.1, 2.2)-Rz] were found to be 0.9 min⁻¹ (±0.12), 0.06 min⁻¹ (±0.015) and 0.042 min⁻¹ (±0.009), respectively. These results indicate that a 2′-amino group at position 2.1 of helix I inhibits ribozyme cleavage activity.

In an attempt to see whether the binding of magnesium was affected by the 2′-amino modification at position 2.1, initial cleavage rates for the Wt-Rz and 2′-NH₂ (2.1)-Rz were determined in the presence of different Mg²⁺ concentrations ranging from 2.5 to 50 mM, since 25 mM is near saturation for Wt-Rz. The cleavage activity of the 2′-NH₂(2.1)-Rz was inhibited by the 2′-amino modification (FIG. 18A). Notably, the cleavage rate of the 2′-NH₂(2.l)-Rz ribozyme hammerhead with Mg²⁺ concentration, suggesting that Mg²⁺ binding is altered by the 2′-amino group at position 2.1. As further illustrated in FIG. 18B, the 2′-NH₂(2.1)-Rz cleaved its substrate with very low efficacy at 2.5 mM Mg²⁺ as compared to the Wt-Rz, thus arguing for a Mg²⁺-related effect.

The 2′-amino Modification did not Alter the Ribozyme/substrate Association

The introduction of a 2′-amino group at position 2.1 may lead to the formation of an unfavourable conformation which may hamper the ribozyme from binding to its substrate. Therefore, we carried out some binding experiments. Various 5′-labelled substrate concentrations were incubated with the same ribozyme concentration and then the ribozyme/substrate complexes were analysed by nondenaturing gels (FIG. 19A). As shown, the binding of the substrate to the ribozyme was not hampered by the selective 2′-amino modification. In the next experiment we have analysed the binding of the substrate as a function of time. Within one min nearly all substrate molecules existed as ribozyme/substrate duplexes for the Wt-Rz and 2′-NH₂(2.1)-Rz (FIG. 19B). Therefore, the binding of the ribozyme to its substrate was not hampered by the 2′-amino modification.

To determine K_{d}, a trace amount of the substrate was incubated with various concentration of each ribozyme for 2 min at 37° C. and then samples were analysed by nondenaturing gels. By plotting the action of the substrate bound versus ribozyme concentration, a K_{d} is determined for all ribozymes. The apparent K_{d} values for Wt-Rz, 2′-NH₂(2.2)-Rz and 2′-NH₂(2.1, 2.2)-Rz were 85 nM (±10), 80 nM (±15), 90 nM (±8), respectively. These values are comparable and therefore the binding results can not explain the drastic decrease in cleavage efficiency of the 2′-NH₂(2.1)-Rz and the 2′-NH2(2.1,2.2)-Rz.

The 2′-amino Modification Alters the Ribozyme Cleavage Step

The data presented above have revealed that the 2′-amino group at position 2.2 and/or 2.1 does not interfere with the ribozyme/substrate association, but it is likely to interfere with Mg²⁺ binding. Since the association step was not significantly affected, we therefore investigated the effect of the 2′-amino modification on the cleavage step. In these experiments, we have used single-turnover conditions. To further assure that the observed cleavage rate represents the actual chemical cleavage step, the 5′-labelled substrate was preannealed to the same large excess of each ribozyme. Following 4 min annealing at 37° C., an aliquot from each sample was analysed immediately by nondenaturing gel to assure that most substrate molecules are complexed with the corresponding ribozyme (FIG. 20A). Cleavage reactions were started by the addition of 10 mM Mg²⁺ (FIG. 20B). Despite nearly complete binding of the substrate to the 2′-amino modified ribozymes as shown in A, only 5% of the substrate was cleaved by the 2′-NH₂(2.1)-Rz following Mg²⁺ addition, while 95% of the substrate was cleaved by the Wt-Rz within 30 seconds. These results suggest that the inhibition arises from a blocking of the cleavage step.

The 2′-amino Modification at Position 2.1 Alters the Global Structure of the Ribozyme/substrate Complexes

The structure of the hammerhead ribozyme complexed with its substrate has been investigated by a number of methods, including crystal structure analysis. Comparative gel electrophoresis has also proved to be a powerful technique in the analysis of the global structures of nucleic acids complexes, including the hammerhead ribozyme. To test the effect of the amino group at position 2.1 on global structure of the ribozyme, an RNA substrate with deoxyribocytosine substitution at C17 to prevent cleavage was designed. The 2′-NH₂(2.1)-Rz and Wt-Rz complexed with the uncleavable susbstrate were analysed by 10% native gel in the presence of 5 mM Mg²⁺ (FIG. 20C). By comparison with the Wt-Rz species, the 2′-NH₂(2.1)-Rz species exhibits a difference in the electrophoretic mobility. The structure of 2′-NH₂(2.1)-Rz/substrate complexes presumably are relaxed, since they are retarded relative to possible compact Wt-Rz/substrate complexes.

Effect of Divalent Metals on Unmodified and Modified Ribozyme Cleavage Activity

The ribozyme cleavage reaction can be facilitated by a variety of divalent metal ions, including Mg²⁺ and Mn²⁺ (Dahm et al. (1991), Biochemistry, 30: 9464-9469). To investigate the effect of Mn²⁺ on ribozyme cleavage activity, various concentrations of Mn²⁺ were tested for their ability to support the Wt-Rz and 2′-NH₂(2.1)-Rz cleavage rates. The reaction conditions were identical to those performed with Mg²⁺. As shown in FIG. 21A, the cleavage rates of the 2′-NH₂ (2.1)-Rz increased dramatically with Mn²⁺.

To see whether the behaviour of 2′-NH₂(2.1)-Rz towards Mn²⁺ is different than that of the Wt-Rz, the acceleration rates with Mn²⁺ vs Mg²⁺ for both ribozymes were calculated (Table 1). A very substantial increase in cleavage rates, especially at low Mg²⁺ concentration was seen with the 2′-NH₂ (2.1)-Rz. In contrast only 0.5 to 2.2 fold increases were obtained with the Wt-Rz. Thus, one can conclude that Mn²⁺ is better able to coordinate to -NH₂ than Mg²⁺. As further illustrated in FIG. 21B, there was a clear difference between the effect of Mn²⁺ upon the unmodified and the modified ribozyme cleavage activity.

The moderate effect of Mn²⁺ upon the Wt-Rz is consistent with some previous studies. For example, Dahm et al., (1991), Biochemistry, 30: 9464-9469; Kuimelis et al., (1996), Biochemistry, 35: 5308-5317.

TABLE 1
Acceleration rates of Mn²⁺ vs Mg²⁺ for Wt-Rz and 2′-
NH₂ (2.1)-Rz

| | |
|---|---|
| Metal ions | vi Mn²⁺/vi Mg²⁺ |

| (mM) | Wt-Rz | 2′-NH₂(2.1)-Rz |
|---|---|---|
| 2.5 | 2.3 | ND |
| 5 | 2.1 | 30 |
| 10 | 1.4 | 12 |
| 25 | 1.6 | 7.3 |
| 50 | 1.5 | 5 |

The acceleration rates are the ratio of the initial cleavage rates in the presence of Mg²⁺ (FIG. 18A) and in the presence of Mn²⁺ (FIG. 21A). ND: indicates that the value can not be determined, because the cleavage activity of the modified ribozyme in the presence of 2.5 mM was extremely low under our cleavage conditions. Although the number of Mg²⁺ ions involved in catalysis by the hammerhead ribozyme and their precise coordination properties remain to be determined, our data suggest the existence of an important Mg²⁺ binding site that was affected by the 2′-amino group at position 2.1. This Mg²⁺ site seems to be required for efficient cleavage, since the addition Mg²⁺ to the preannealed 2′-NH₂ (2.1)-Rz-substrate complexes did not enhance cleavage when compared to the Wt-Rz. However, in contrast to Mg²⁺, addition of Mn²+to the preannealed 2′-NH₂(2.1)-Rz/substrate duplexes resulted in efficient cleavage of the substrate (FIG. 21C).

For comparison, we have also investigated the ability of the Wt-Rz and the 2′-NH₂(21)-Rz to cleave in the presence of other series of divalent metals (FIG. 22). In contrast to Mn²⁺, none of the tested metals rescued the modified ribozyme cleavage activity. In the case of Co²⁺ and Ca²⁺, there is inhibition of the Wt-Rz cleavage activity when compared to Mg²⁺. Notably, the binding of Co²⁺ and Ca²⁺ to the ribozyme seems to be affected by the 2′-amino modification, since they are not used effectively as catalytic cofactors by the ribozyme. Since the thiophilic metal ions Co²⁺ did not promote the rescue of the 2′-NH₂(2.1)-Rz, so it appears that in the present case the rescue ability of Mn²⁺ is specific.

Implication of Our Findings on the Mechanism of Ribozyme Cleavage

Several models have been proposed for the cleavage of RNA phosphodiester bonds by hammerhead ribozyme, basically a one-metal-hydroxyde ion model and a two-metal-ion model. In a one-metal ion mechanism, a solvated single metal hydroxyde coordinates to the pro-R oxygen of the phosphate and acts as a base to abstract a proton from the 2′OH of nucleotide 17. The activated 2′-O⁻ then acts as a nucleophile by attacking the phosphate and displacing the 5′-oxygen of the leaving base. The two-metal-ion mechanism is the same as the one-metal-ion model, except that a second metal coordinates to the 5′-oxygen of the leaving base. To identify potential oxygen atoms that interact with Mg²⁺, several phosphate oxygens in the substrate have been replaced by thiol groups. These experiments are based upon the fact that oxygen atoms bind soft metal and hard metal with similar affinity, while sulfur atoms bind soft metal ions more strongly than hard metal ions. An enhancement of the ribozyme cleavage rate in presence of Mn²⁺ relative to Mg²⁺ was explained with the double mechanism of catalysis. However, in other cases the cleavage rates of a ribozyme in presense of Mg²⁺ and Mn²⁺ were found to be almost identical. Therefore the absence of an accelerated rate would support a one-metal-ion mechanism of catalysis.

The data presented in this Example support the existence of a potential Mg²⁺ binding site which is more likely to be disrupted by the presence of 2′-amino group at position 2.1. This Mg²⁺ binding site could participate in the conformation of an active transition state, since preannealed substrate to 2′-NH₂ (2.1)-Rz was not effectively cleaved by Mg²⁺ addition, and since an altered global structure of the 2′-NH₂(2.1)-Rz in the presence of Mg²⁺ was detected by native gel electrophoresis (FIG. 20C).

Notably, the crystallographic data reported by Pley et al., (1994), Nature, 372: 68-74 of the hammerhead ribozyme complexed with DNA substrate have revealed a single Mg²⁺ binding site near the cleavage site. However, the recent X-ray by Scott et al., (1995), Cell, 81: 991-1002; Scott et a., (1996), Science, 274: 2065-2069, shows the presence of at least 5 binding sites for Mg²⁺ or Mn²⁺. Further analysis indicates the presence of multiple binding sites for Co²⁺ and Zn²⁺ (Murray et al., (1998), Cell, 92: 665-673). In all hammerhead ribozyme X-ray crystal structures, in particular the most advanced one reported by Murray et al., local structural changes seem to be required for the in-line cleavage. Such changes are more likely to be initiated by binding of Mg²⁺ to critical RNA sites. In this connection the binding of Mg²⁺ close to the cleavage site was found to induce conformational change of ribozyme-substrate complexes (Mengel et al., (1996), Biochemistry 35: 14710-16). Our data would support these findings.

Interestingly, the inhibitory effect of a 2′-amino group at position 2.1 on the ribozyme cleavage rate is substantially rescued by replacement of Mg²⁺ by Mn²⁺ ions. Furthermore and in contrast to Mg²⁺, Mn²⁺ was found to promote quickly the cleavage of preannealed 2′-modified ribozyme substrate (FIG. 21C). In some studies, Mn²⁺ was found to rescue the cleavage of modified substrates by hammerhead ribozymes (Pontius et al., 91997), Proc. Natl. Acad. Sci. USA, 94: 2290-2294). In the case of phosphorothioate substrates, the Mn²⁺ effect was due to its ability to efficiently coordinate with the 5′ sulfur leaving group. In the present study, the rescue effect of Mn²⁺ on the 2′-NH₂(2.1)-Rz may originate for its ability to coordinate to the -NH₂ group or from its ability to bind to another metal binding site. The binding to such a site may affect the global structure of the ribozyme and therefore eliminates any potential steric hindrance introduced by the amino group at position 2.1. Recently, it was demonstrated that in its most active state, the ribozyme is populated by several Mn²⁺ ions with different degrees of affinity (Horton et al., (1998), Biochemistry, 37: 18094-18101). Surprisingly, two other thiophilic metal ions Co²⁺ and Cd²⁺ did not restore the 2′-NH₂(2.1)-Rz cleavage activity, suggesting that the thiophilic properties of the metal is not sufficient for the rescue effect observed in the present study.

Conclusions

Taken together, the results presented here indicate that the presence of an amino group at position 2.1 inhibits ribozyme cleavage step by more likely interfering with Mg²⁺ binding. The demonstration that the global structure of the modified ribozyme/substrate complexes in the presence of Mg²⁺ is different from the complexes formed with its unmodified version would support the notion that the binding of Mg²⁺ is affected by the 2′-amino modification. In contrast to Mg²⁺, Mn²⁺ substantially rescued the ribozyme cleavage activity, suggesting that it can coordinate to -NH₂ group. These observations are novel and should facilitate the chemical modification of ribozymes. In addition, ribozymes with a 2′-amino group at position 2.1 may represent a novel tool to investigate the mechanistic model, the role of metal ions in hammerhead ribozyme catalysis and to trap conformational intermediates during crystallography.

1. A modified hammerhead ribozyme wherein at least 90% of the pyrimidine bases therein are 2′-amino modified, wherein base 2.1, and optionally base 2.2, are purine bases and having at least 20% catalytic activity of the unmodified ribozyme.

2. A modified ribozyme as claimed in claim 1 having at least 50% catalytic activity of the unmodified ribozyme.

3. A modified ribozyme as claimed in claim 2 having at least 80% catalytic activity of the unmodified ribozyme.

4. A modified ribozyme as claimed in any preceding claim, wherein said modified ribozyme exhibits 90% or more of the catalytic activity of the unmodified ribozyme.

5. A modified ribozyme as claimed in claim 1, wherein all of the pyrimidine nucleotides which are present in said ribozyme are 2′-amino modified.

6. A modified ribozyme as claimed in any preceding claim, wherein less than 50% of the bases present in the ribozyme to be modified are pyrimidines.

7. A modified ribozyme as claimed in any preceding claim wherein less than 30% of the ribonucleotides in helix 1 are pyrimidines.

8. A modified ribozyme as claimed in any preceding claim wherein bases 2.1, 2.2 and 15.2 are purine bases.

9. A modified ribozyme as claimed in any preceding claim, wherein said ribozyme is 2′-amino modified at at least positions 4 and 7.

10. A modified ribozyme as claimed in any one of the preceding claims, wherein said ribozyme comprises a conserved central portion flanked by binding site recognition sequences and wherein said central portion has the sequence 5′ CUGANGA(N)ₓNNNN(N′)ₓGAAA 3′, wherein N represents A, C, G or U; x is 2, 3, 4 or 5; and N′ represents a ribonucleotide, ie. A, C, G or U, such that (N′)ₓ is complementary to (N)ₓ to allow the formation of Watson-Crick hydrogen bonding.

11. A modified ribozyme as claimed in any preceding claim, wherein said ribozyme is derived from any one of the following sequences:

| | |
|---|---|
| 5′-GAAGGCCGGGUACUGAUGAGUCCGUGAGGACGAAACGUCAGCCAU-3′ | SEQ ID NO. 1 |
| (PKCα ribozyme); | |
| 5′-GAAGAGGCUGACUGAUGAGUCCGUGAGGACGAAACAUAGGCACCG-3′ | SEQ ID NO. 2 |
| (TNFα ribozyme); | |
| 5′-GGGAAGGCCGGGAACUGAUGAGUCCGUGAGGACGAAACGUCAGCCAU-3′ | SEQ ID NO. 10 |
| 5-GGGGGAACUGAUGAGUCCGUGAGGACGAAACGUCAGCCAU-3′ | SEQ ID NO. 3 |
| (human PKCα ribozyme); | |
| 5′-GGAAAGACUGAUGAGUCCGUGAGGACGAAAGCAGAAAGUGCAUGG-3′ | SEQ ID NO. 4 |
| (rat VEGF ribozyme); and | |
| 5′-GAGCAGACUGAUGAGUCCGUGAGGACGAAAGUUCAUGG-3′ | SEQ ID NO. 5 |
| (human VEGF ribozyme), | |

or a sequence having at least 70% sequence homology to any aforesaid sequence, or which hybridises to a complement of a said sequence under conditions of high stringency,
or a functionally equivalent analog, variant or fragment thereof.

12. A ribozyme as claimed in any one of claims 1 to 11 for use in therapy.

13. A ribozyme as claimed in any one of claims 1 to 12 for treating cancer.

14. Use of a ribozyme as defined in any one of claims 1 to 11 in the manufacture of a medicament for treating or preventing a disease or condition responsive to an alteration in the expression of a gene wherein said ribozyme is capable of cleaving the RNA transcribed from said gene.

15. A method of treating or preventing a disease or condition responsive to an alteration in the expression of a gene, said method comprising administering a ribozyme as defined in any one of claims 1 to 11, wherein said ribozyme is capable of cleaving the RNA transcribed from said gene.

16. A method or use as claimed in claim 14 or 15 wherein said disease or condition is associated with the proliferation of rapidly dividing cells.

17. A method or use as claimed in claim 14 or 15 wherein said disease or condition is cancer.

18. A method or use as claimed in claim 14 or 15 wherein said disease or condition is a malignant glioma.

19. An in-vitro method for inhibiting proliferation of cells comprising contacting said cells with a ribozyme as defined in claim 11.

20. Use of a ribozyme as defined in claim 10 or claim 11 for inhibiting the proliferation of cells.

21. The use, method of treatment or in-vitro method of any one of claims 16 to 20 wherein said ribozymes are catalytically-inactive or exhibit reduced catalytic activity.

22. A pharmaceutical composition comprising a ribozyme as defined in any one of claims 1 to 11 together with at least one pharmaceutically acceptable carrier or excipient.

23. A pharmaceutical composition as claimed in claim 22, further comprising a biologically active agent.

24. Use of a ribozyme as defined in any one of claims 1 to 11 in hydrolysing RNA or as an antisense molecule.
